(19) United States
(12) Patent Application Publication
(10) Pub. No.: US 2014/0315720 A1
(43) Pub. Date: Oct. 23, 2014

(54) POLYSACCHARIDE ESTER MICROSPHERES AND METHODS AND ARTICLES RELATING THERETO

(71) Applicant: Celanese Acetate LLC, Irving, TX (US)

(72) Inventors: Denis G. Fallon, Blackburg, VA (US); Thomas S. Garrett, Narrows, VA (US); Lawton E. Kizer, Blacksburg, VA (US); Karen L. Zazzara, Blacksburg, VA (US); Michael T. Combs, Blacksburg, VA (US); Richard K. Johnson, Blacksburg, VA (US); Gary Dehart, Paterstown, WV (US)

(73) Assignee: Celanese Acetate LLC, Irving, TX (US)

(21) Appl. No.: 14/244,998

(22) Filed: Apr. 4, 2014

Related U.S. Application Data
(63) Continuation of application No. 14/060,782, filed on Oct. 23, 2013.
(60) Provisional application No. 61/717,726, filed on Oct. 24, 2012.

(51) Int. Cl. A01N 25/34; A24D 3/14; A23L 1/03; A61K 8/02

(52) U.S. Cl. CPC A01N 25/34; A61K 8/025; A24D 3/14; A23L 1/0315
USPC 504/358; 424/401; 424/63; 424/64; 424/405; 131/332; 428/402; 426/661

(57) ABSTRACT

A method for producing a polysaccharide ester microsphere may include forming a polysaccharide ester product from a polysaccharide synthesis, wherein the polysaccharide ester product comprises a polysaccharide ester and a solvent; diluting the polysaccharide ester product, thereby yielding a polysaccharide ester dope; and forming a plurality of polysaccharide ester microspheres from the polysaccharide ester dope. Suitable polysaccharides may include, but are not limited to, starch, cellulose, hemicellulose, algenates, chitosan, and any combination thereof. Esters thereof may be organic esters (e.g., acetate and the like), inorganic esters (e.g., sulfonates and the like), or combinations thereof. Further, the solids conent of the polysaccharide ester dope, in some instances, may be greater than about 16 wt %.

### CROSS-REFERENCE TO RELATED APPLICATIONS

This application is a continuation of U.S. patent application Ser. No. 14/060,782 by Denis G. Fallon, filed on Oct. 23, 2013, entitled Polysaccharide Ester Microspheres and Methods and Articles Relating Thereto, which claims priority to U.S. Provisional Patent Application No. 61/717,726.

### BACKGROUND

The exemplary embodiments described herein relate to polysaccharide ester microspheres and methods and articles relating thereto.

The most common polysaccharide ester to be used in microspheres is cellulose ester. Cellulose ester microspheres are often substantially rigid beads that consist essentially of cellulose ester and have some degree of porosity based on the method with which they are produced. Cellulose ester microspheres have proven useful for immobilizing enzymes and release of chemicals, especially in conjunction with filter and chromatograph packing materials. Further, cellulose ester microspheres have found some industrial success in pharmaceutical and cosmetic applications. However, these applications, in addition to food, personal care, agricultural applications, and others, would benefit from and/or be further enabled with the ability to produce cellulose ester microspheres at lower cost and with tailored properties (e.g., release rate of additives, solubility, polarity, and strength).

Methods for forming cellulose ester microspheres include solvent evaporation methods, precipitation methods, and emulsion methods that utilize cellulose ester flake as the precursor. The production of cellulose ester flake can be energy intensive, especially in the finishing process that yield the dry flake after the reactions are completed. Further, these production methods generally provide for batch production of cellulose ester microspheres, which increases production cost, time, and waste. For industrial uses to be a reality, a continuous production process is desirable to reduce cost and increase product uniformity.

Additionally, tailoring the properties of the cellulose ester microspheres may enable a wider breadth of applications than have been previously realized. For example, cosmetics are increasingly using controlled release to deliver vitamins and minerals and/or release fragrances over moderate time periods, like a day. Similarly, additives for agricultural applications (e.g., fertilizers and pesticides) are increasingly being investigated for controlled and/or long-term efficacy with single or minimal treatments. Cellulose ester microspheres have been limited in the ability to adapt the properties to a specific additive in combination with an application. For example, a desired release rate may be similar between a vitamin in a cosmetic application and a pesticide in an agricultural application, but the hardness and size for the respective applications may be very different. Therefore, multi-dimensional tailoring of properties and production on an industrial-scale may be of value in expanding the practical applications of cellulose ester microspheres.

### BRIEF DESCRIPTION OF THE DRAWINGS

The following figures are included to illustrate certain aspects of the exemplary embodiments described herein, and should not be viewed as exclusive embodiments. The subject matter disclosed is capable of considerable modifications, alterations, combinations, and equivalents in form and function, as will occur to those skilled in the art and having the benefit of this disclosure.

FIG. 1 provides nonlimiting examples of methods for forming microspheres according to at least some embodiments described herein.

FIG. 2 provides an uptake profile for a cellulose ester microsphere according to at least some embodiments described herein.

FIG. 3 provides SEM micrograph of several microspheres produced according to at least some embodiments described herein.

FIG. 4 provides SEM micrograph of several microspheres produced according to at least some embodiments described herein.

FIG. 5 provides SEM micrograph of several microspheres produced according to at least some embodiments described herein.

FIG. 6 provides SEM micrograph of several microspheres produced according to at least some embodiments described herein.

FIG. 7 provides SEM micrograph of several microspheres produced according to at least some embodiments described herein.

FIG. 8 provides SEM micrograph of several microspheres produced according to at least some embodiments described herein.

FIG. 9 provides SEM micrograph of several microspheres produced according to at least some embodiments described herein.

FIG. 10 provides SEM micrograph of several microspheres produced according to at least some embodiments described herein.

FIG. 11 provides SEM micrograph of several microspheres produced according to at least some embodiments described herein.

FIG. 12 provides SEM micrograph of several microspheres produced according to at least some embodiments described herein.

FIG. 13 provides SEM micrograph of several microspheres produced according to at least some embodiments described herein.

FIG. 14 provides SEM micrograph of several microspheres produced according to at least some embodiments described herein.

FIG. 15 provides SEM micrograph of several microspheres produced according to at least some embodiments described herein.

### DETAILED DESCRIPTION

The exemplary embodiments described herein relate to polysaccharide ester microspheres and methods and articles relating thereto.

As used herein, the term “microsphere” encompasses shapes that are spherical, substantially spherical, oblong, substantially oblong, teardrop, substantially teardrop, prolate spherical, substantially prolate spherical, and the like, and any hybrid thereof.

The polysaccharide ester microspheres described herein comprise at least one polysaccharide ester. The compositional versatility of polysaccharide esters and ability to mix various polysaccharide esters may allow for tailoring the properties (e.g., size, strength, morphology, solubility, and polarity) of the polysaccharide ester microspheres, which may be useful in expanding the applications of polysaccharide ester microspheres. For example, tailoring the properties of the polysaccharide ester microspheres may allow for manipulating the release rates of additives (e.g., pharmaceuticals, flavors, nutraceuticals, plant nutrients, and insect repellents) from the polysaccharide ester microspheres.

Further, described herein are methods that facilitate industrial production of polysaccharide ester microspheres with tailorable properties. For example, continuous production methods may be employed to reduce the time, cost, and waste associated with their production and facilitates their use in industrial applications. Additionally, in some embodiments, the polysaccharide ester microspheres may be produced directly from a dope extracted from the production of the polysaccharide ester. That is, at least some polysaccharide ester microsphere production methods do not require a finished form (e.g., flake) of the polysaccharide ester. By not having to finish the polysaccharide ester, the associated time, cost, energy consumption, and chemical waste is reduced. Accordingly, the polysaccharide ester microsphere production methods described herein may, in some embodiments, advantageously have a significantly smaller environmental impact while being cost effective for industrial scale-up.

It should be noted that when “about” is used herein in reference to a number in a numerical list, the term “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

### I. Polysaccharide Ester Microsphere Compositions

Examples of suitable polysaccharide esters may include, but are not limited to, ester derivatives of starch, cellulose, hemicellulose, algenates, chitosan, and the like, and any combination thereof. Ester derivatives may be organic esters substituents, inorganic ester substituents, or a combination thereof.

As used herein, the term “starch” refers to a natural polysaccharide that includes amylose and amylopectin in various ratios and derivatives thereof. Example of starches may include, but are not limited to, waxy starches, modified starches, native starches, dextrins, and maltodextrins with dextrose equivalents of 1-50. Suitable starch sources for starch esters may, in some embodiments, include, but are not limited to, cereals, rice, wheat, maize, root vegetables, potatoes, corn, tapioca, cassava, acorns, arrowroot, arracacha, bananas, barley, breadfruit, buckwheat, canna, colacasia, katakuri, kudzu, malanga, millet, oats, oca, polynesian arrowroot, sago, sorghum, sweet potatoes, rye, taro, chestnuts, water chestnuts, yams, beans, favas, lentils, mung beans, peas, chickpeas, and the like, and any combination thereof.

Suitable cellulosic sources for cellulose esters may, in some embodiments, include, but are not limited to, softwoods, hardwoods, cotton linters, switchgrass, bamboo, bagasse, industrial hemp, willow, poplar, perennial grasses (e.g., grasses of the Miscanthus family), bacterial cellulose, seed hulls (e.g., soy beans), and the like, and any combination thereof.

The organic ester substituent may include, but are not limited to, C₁-C₂₀ aliphatic esters (e.g., acetate, propionate, or butyrate), functional C₁-C₂₀ aliphatic esters (e.g., acrylates or diesters), aromatic esters (e.g., benzoate or phthalate), substituted aromatic esters, and the like, any derivative thereof, and any combination thereof. Without being limited by theory, it is believed that the organic ester substituent of the polysaccharide ester described herein may affect, inter alia, the release rate of an additive from a polysaccharide ester microsphere described herein.

In some embodiments, the degree of substitution of the polysaccharide ester may range from a lower limit of about 0.2, 0.4, 0.6, 0.8, 1, 1.5, 2, 2.3, or 2.5 to an upper limit of less than about 3, 2.9, 2.7, 2.5, 2, or 1.5, and wherein the degree of substitution may range from any lower limit to any upper limit and encompass any subset therebetween. As used herein, the term “degree of substitution” refers to the average number of hydroxyl groups that have been replaced by organic functional groups bound to an anhydroglucose unit, which can be determined by a plurality of methods including chemical saponification and titration, liquid chromatography, NIR, and the like. In some embodiments, a polysaccharide ester may be cellulose diacetate having a degree of substitution ranging from about 2.2 to about 2.7. In some embodiments, a polysaccharide ester may be cellulose triacetate having a degree of substitution of about 2.7 or greater.

As used herein, the term “inorganic ester substituent” refers to an ester that comprises an oxygen bound to an R group and an inorganic, nonmetal atom (e.g., sulfur, phosphorus, boron, and chlorine). It should be noted that inorganic esters encompass esters derived from oxoacids that comprise both inorganic, nonmetal atoms and carbon atoms (e.g., alkyl sulfonic acids like methane sulfonic acid). Inorganic ester substituents may include, but are not limited to, hypochlorite, chlorite, chlorate, perchlorate, sulfite, sulfate, sulfonates (e.g., taurine, toluenesulfonate, C₁-C₁₀ alkyl sulfonate, and aryl sulfonate), fluorosulfate, nitrite, nitrate, phosphite, phosphate, phosphonates, borate, and the like, any derivative thereof, and any combination thereof.

In some embodiments, the weight percent of the inorganic, nonmetal atom of the inorganic ester substituent of a polysaccharide ester may range from a lower limit of about 0.01%, 0.05%, or 0.1% to an upper limit of about 8%, 5%, 3%, 1%, 0.5%, 0.25%, 0.2%, or 0.15%, and wherein the weight percent may range from any lower limit to any upper limit and encompass any subset therebetween.

Polysaccharide esters with inorganic ester substituents may be produced via several synthesis routes, at least some of which are described in further detail in copending International Patent Application No. PCT/US12/56802 entitled “Substituted Cellulose Ester Adhesives and Methods and Articles Relating Thereto” filed on Sep. 24, 2012, the entire disclosure of which is incorporated herein by reference.

Tailoring the properties of the polysaccharide ester microspheres described herein (e.g., average particle size, particle size distribution, porosity, solubility, and hardness) may be achieved, in some embodiments, by tailoring the properties of the at least one polysaccharide ester and, if more than one, the relative ratios thereof. Properties of the polysaccharide esters that may, in some embodiments, be useful to tailor may include, but are not limited to, the organic ester substituent, degree of substitution, composition and amount of additional substituents (e.g., inorganic ester substituents), molecular weight, solubility, hydrophobicity, and the like, and any combination thereof.

In some embodiments, the polysaccharide ester microspheres described herein may comprise two or more types of polysaccharide esters. As used herein, types of polysaccharide esters may be differentiated by at least one property of the polysaccharide ester (e.g., composition and amount of organic ester substituent(s), composition and amount of inorganic ester substituent(s), molecular weight, composition of the polysaccharide, and the like). In some embodiments where two or more types of polysaccharide esters are used, the amount of each polysaccharide ester may independently range from a lower limit of greater than 0%, about 1%, 10%, 25%, or 50% by weight of the total polysaccharide esters to an upper limit of about 99%, 90%, 75%, or 50% by weight of the total polysaccharide esters, and wherein the amount of each polysaccharide ester may independently range from any lower limit to any upper limit and encompass any subset therebetween.

Without being limited by theory, it is believed that the chemical composition the polysaccharide esters (e.g., the composition and degree of substitution of an organic substituent and the composition and amount of an inorganic substituent) may affect, inter alia, the release rate of an additive (e.g., a dye, a pharmaceutical, a nutraceutical, a pesticide, and the like) from a polysaccharide esters microsphere, the uptake rate of an additive (e.g., a dye, a contaminant, and the like) into a polysaccharide esters microsphere, the crystallinity of a polysaccharide esters microsphere, the hydrophobicity of a polysaccharide esters microsphere, and the solubility of a polysaccharide esters microsphere. By way of nonlimiting example, polysaccharide esters microspheres comprising a polysaccharide ester with a degree of substitution of about 2.7 or greater may have a high crystallinity and a reduced rate of release for an additive disposed therein as compared to a polysaccharide ester having a degree of substitution of about 2.2 to about 2.7. By way of another nonlimiting example, the properties of the polysaccharide ester microspheres may be tailored by adjusting the ratio two or more polysaccharide esters. By way of yet another nonlimiting example, lower degrees of substitution may allow for additives capable of hydrogen bonding to diffuse more slowly through the polysaccharide ester and, consequently, release from a polysaccharide esters microsphere at a lower rate than other additives.

In some embodiments, the average molecular weight of a polysaccharide ester described herein may be about 1,000,000 g/mol or less, about 100,000 g/mol or less, or more preferably about 50,000 g/mol or less. In some embodiments, the average molecular weight of a polysaccharide ester may range from a lower limit of about 1,000 g/mol, 5,000 g/mol, 10,000 g/mol, 25,000 g/mol, 50,000 g/mol, or 100,000 g/mol to an upper limit of about 1,000,000 g/mol, 500,000 g/mol, 250,000 g/mol, 100,000 g/mol, or 50,000 g/mol, and wherein the average molecular weight may range from any lower limit to any upper limit and encompass any subset therebetween. As used herein, the term “average molecular weight” refers to the number average molecular weight as determined by gel permeation chromatography using a polystyrene standard.

In some embodiments, the polysaccharide ester may be soluble in water, acetone, acetic acid, methylene chloride, dimethyl sulfoxide, dimethyl carbonate, dimethyl formamide, N-methylpyrrolidone, mixtures thereof, and the like. As used herein, the term “soluble” refers to a substantially dissolved, single phase, and homogeneous substance that may have a small fraction (e.g., less than about 1%) of the substance being a semi-soluble (e.g., a gel). One skilled in the art will know that the degree of substitution and the composition and amount of inorganic ester substituents affects the solvent selection. By way of nonlimiting example, a polysaccharide ester (e.g., cellulose acetate) with a degree of substitution of about 0.4 to about 1.2 may be soluble in water. By way of another nonlimiting example, a polysaccharide ester having a degree of substitution of about 0.7 to about 2.7 and a sulfate inorganic ester substituent with about 0.006% to about 5% sulfur by weight may be soluble in a mixed solvent that comprises an aqueous solvent and an organic solvent (e.g., acetone).

In some embodiments, the hydrophobicity of the polysaccharide esters may be modified or tuned to affect final material properties. Hydrophobicity may be measured by forming a film of the polysaccharide ester and measuring the contact angle of water thereon.

Tailoring the properties of the polysaccharide esters described herein may be achieved, in some embodiments, through adjustment in, inter alia, the conditions of synthesis, the polysaccharide material from which the polysaccharide esters are synthesized, and any combination thereof.

### II. Methods of Producing Polysaccharide Ester Microspheres

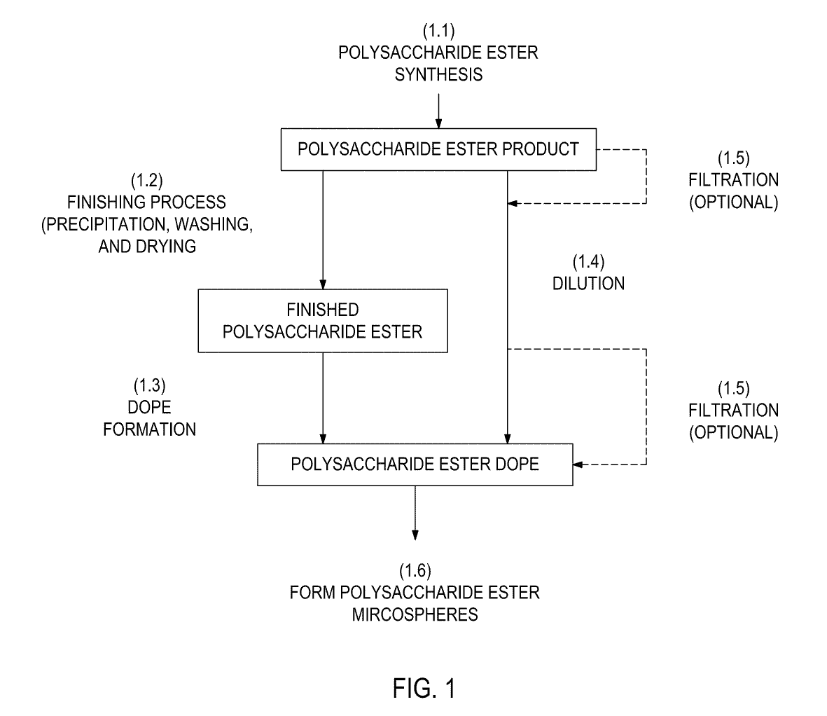

Referring to FIG. 1, in some embodiments, forming polysaccharide ester microspheres described herein may proceed by a series of reactions that include synthesizing the polysaccharide ester (1.1), which may be by a variety of methods depending on the desired composition, to yield a polysaccharide ester product. The polysaccharide ester product may then be processed in one of two general methods. The first method includes the more traditional preparation method of a finishing process (1.2) that precipitates the polysaccharide ester product from the solvent of the synthesis reaction (e.g., a mixture of acetic acid and water), washing the precipitated polysaccharide ester, and drying the polysaccharide ester into a finished polysaccharide ester, which may be in a powder or flake form, for example. Then, in dope formation (1.3), the finished polysaccharide ester may then be dissolved into a solvent to yield a polysaccharide ester dope. The polysaccharide ester dope may then be formed into polysaccharide ester microspheres (1.6).

Alternatively, the polysaccharide ester product may be formed directly into a dope by dilution (1.4) of the polysaccharide ester product from the polysaccharide ester synthesis (1.1). Optionally before or after dilution, the solution may be filtered (1.5) (optionally with the addition of filter aids) to remove, for example, unreacted polysaccharide and salts formed during the polysaccharide ester synthesis (1.1). The polysaccharide ester dope may then be formed into polysaccharide ester microspheres (1.6).

It should be noted that polysaccharide ester dopes may be produced with more than one polysaccharide ester described herein by adding two or more polysaccharide esters together during any stage between polysaccharide ester synthesis (1.1) and polysaccharide ester microsphere formation (1.6). Further, the individual polysaccharide esters in embodiments with two or more polysaccharide esters in a polysaccharide ester dope may be produced and/or treated by any suitable methods including those described herein. For example, a first polysaccharide ester may be synthesized and then treated by a finishing process (1.2) and a dope formation (1.3), while a second polysaccharide ester may be synthesized and diluted (1.4) with the polysaccharide ester dope comprising the first polysaccharide ester. In another example, two polysaccharide ester products may be mixed, filtered (1.5), diluted (1.4), and filtered (1.5) again to yield a polysaccharide ester dope.

Referring to the dope formation process (1.3) and the dilution process (1.4), suitable dope solvents may include, but are not limited to, acetic acid, water, methanol, ethanol, propanol, acetone, nitromethane, dioxane, tetrahydrofuran, pyridine, methyl ethyl ketone, dimethyl sulfoxide, methyl acetate, dichloromethane, chloroform, tetrachloroethane, trichloroethane, dimethyl sulfoxide, dimethyl formamide, dimethyl carbonate, ethylene carbonate, propylene carbonate, and the like, and any combination thereof.

In some embodiments, the dope formation process (1.3) and the dilution process (1.4) may also utilize a non-solvent that may assist in forming the polysaccharide ester microspheres. In some embodiments, suitable dope non-solvents may include, but are not limited to, acetic acid, water, methanol, ethanol, propanol, acetone, nitromethane, dioxane, tetrahydrofuran, pyridine, methyl ethyl ketone, dimethyl sulfoxide, dimethyl carbonate, propylene carbonate, ethylene carbonate, methyl acetate, dichloromethane, chloroform, tetrachloroethane, trichloroethane, and the like, and any combination thereof. One skilled in the art with the benefit of this disclosure should recognize that the properties of the polysaccharide ester product will, inter alia, determine suitable dope solvents.

By way of nonlimiting example, polysaccharide esters having a degree of substitution of about 2.5 may utilize acetone as a solvent and water as a non-solvent in the polysaccharide ester dope, where polysaccharide esters having high degrees of substitution may utilize acetic acid as the solvent and water as the non-solvent. In some embodiments, a non-solvent may be present in a polysaccharide ester dope in an amount ranging from a lower limit of 0%, about 0.1%, 1%, 5%, or 10% by weight of the solvent to an upper limit of about 40%, 30%, 20%, 15%, or 10% by weight of the solvent, and wherein the amount of non-solvent may range from any lower limit to any upper limit and encompass any subset therebetween

In some embodiments, the polysaccharide ester dope described herein may have a solids concentration of about 50% by weight or less, about 30% by weight or less, or about 25% by weight or less. In some embodiments, the polysaccharide ester dope described herein may have a solids concentration ranging from a lower limit of about 4%, 6%, 8%, 10%, 12%, or 15% to an upper limit of about 50%, 40%, 30%, 25%, 18%, 16%, 14%, or 12% by weight, and wherein the solids concentration may range from any lower limit to any upper limit and encompasses and subset therebetween.

Referring to the filtration process (1.5), filtration may, in some embodiments, remove unwanted components, unreacted polysaccharide material, reaction byproducts, and any combination thereof from the polysaccharide ester product. For example, salts like magnesium sulfate, calcium sulfate, or ammonium sulfate may, in some embodiments, be produced in the esterification and/or hydrolysis reactions. Further, in some embodiments, unreacted polysaccharide material (e.g., cellulose fibers) may also be removed from a polysaccharide ester product described herein via filtration. As described above, the filtration process (1.5) is optional. For example, with some dissolving-grade cellulose starting materials, the polysaccharide ester synthesis (1.1) may be sufficiently clean to produce a suitable polysaccharide ester dope. One skilled in the art with the benefit of this disclosure should recognize that the need for and amount of filtration may be influenced by, inter alia, the polysaccharide starting material, the desired purity of the polysaccharide ester microspheres, the application of the polysaccharide ester microspheres, the desired properties of the polysaccharide ester microspheres, and the like.

Referring to the microsphere forming process (1.6), forming polysaccharide ester microspheres described herein may, in some embodiments, involve forming droplets of a polysaccharide ester dope and coagulating the droplets into microspheres in a coagulation bath. In some embodiments, a coagulation bath may comprise a non-solvent as described above in relation to the dope formation process (1.3) and the dilution process (1.4). It should be noted that the non-solvent utilized to form the polysaccharide ester dope and the non-solvent of the coagulation bath may be the same or different.

In some embodiments, forming droplets may involve methods that include, but are not limited to, spheronizing, aerosol spraying, airless spraying, ultrasonic spraying, rotating disk spraying, dripping, impinging on rotating atomizer plates, and the like, any hybrid thereof, and any combination thereof. In some embodiments, such methods may utilize double nozzles that enable formation of core-shell microspheres. In some embodiments, the core and/or the shell of a core shell microspheres may comprise a polysaccharide ester described herein.

In some embodiments, the polysaccharide ester dope may comprise salts, which may affect the morphology, surface roughness, diameter, and the like of the polysaccharide ester microspheres produced. In some instances, the salts may be present as a result of using a polysaccharide ester product without finishing. In some instances, the salts may be added to the polysaccharide ester dope. A hybrid of the foregoing is also envisioned.

Examples of salts may include, but are not limited to, sodium, potassium, magnesium, calcium, and the like salts of inorganic acids or organic acids, including combinations thereof (e.g., chlorides, bromides, sulfates, phosphate, acetates, formates, and the like).

In some embodiments, a coagulation bath may comprise surfactants that participate in the formation of the polysaccharide ester microspheres and may affect the morphology and/or diameter of the polysaccharide ester microspheres produced. In some embodiments, a polysaccharide ester dope may comprise surfactants that participate in the formation of the polysaccharide ester microspheres and may affect the morphology and/or diameter of the polysaccharide ester microspheres produced. Inclusion of surfactants in the polysaccharide ester dope may improve raw material usage or product uniformity.

Examples of surfactants may, in some embodiments, include, but are not limited to, TWEENS® (polyoxyalkylene derivatives of hexitol anhydride partial long chain fatty acid esters, available from SigmaAldrich), SPANS® (partial esters of common fatty acids such as caloric, palmitic, stearate, and oleic acids, and hexitol anhydrides, available from SigmaAldrich), NIA-PROOF® #4 (sodium tetradecyl sulfate, available from Niacet Corporation), polyethylene glycols, ethylene glycol-propylene glycol-ethylene glycol triblock copolymers, fatty alcohol ethoxylates, C₅-C₂₀ sulfates, C₅-C₂₀ sulfonates, C₅-C₂₀ phosphates, C₅-C₂₀ carboxylates, TRITONS® (surfactants with hydrophilic polyethylene oxide chains and hydrophobic aromatic hydrocarbons, available from SigmaAldrich), fatty acid ethoxylates, aromatic acid ethoxylates, and the like, any derivative thereof, and any combination thereof.

In some embodiments, the distance between droplet formation and the coagulation bath may affect the morphology and/or porosity of the polysaccharide ester microspheres produced. For example, the distance between droplet formation and the coagulation bath may, in some embodiments, range from about 20 inches to about 100 inches.

In some embodiments, the temperature of the microsphere forming process (1.6) (including portions thereof like the dope temperature, ambient temperature, and coagulation bath temperature) may also affect the morphology and/or porosity of the polysaccharide ester microspheres produced. For example, the temperature of dope and/or the coagulation bath may, in some embodiments, independently range from about 15° C. and about 90° C. Further, in some embodiments, the temperature of the dope and the coagulation bath may be the same or different.

After formation, the polysaccharide ester microspheres may be removed from the coagulation bath, washed, and dried. In some embodiments, the microsphere forming process (1.6) and subsequent washing and drying processes may be a continuous process, a batch process, or a hybrid thereof. For example, polysaccharide ester microspheres may be continuously formed and transferred continuously from the coagulation bath, e.g., by conveyor, through areas that provide washing and drying of the polysaccharide ester microspheres. Continuous processing may advantageously reduce both labor and manufacturing costs and, in some instances, increase the production speed. Further, in some embodiments, continuous processes may have at various points along the process quality control measures enacted.

Properties of the polysaccharide ester microspheres that may, in some embodiments, be useful to tailor may include, but are not limited to, porosity, crystallinity, crush strength, surface area, particle size, dye uptake, polarity, hydrophobicity, release rate of an additive from the polysaccharide ester microspheres, morphology, degradation, and the like, and any combination thereof.

In some embodiments, the surface area of the polysaccharide ester microspheres may range from a lower limit of about 1 m²/g, 5 m²/g, 10 m²/g, or 15 m²/g to an upper limit of about 50 m²/g, 40 m²/g, 30 m²/g, or 25 m²/g, and wherein the surface area may range from any lower limit to any upper limit and encompass any subset therebetween. The surface area may be measured with BET analysis with nitrogen gas.

In some embodiments, the total pore volume of the polysaccharide ester microspheres may range from a lower limit of about 0.05 mL/g, 0.07 mL/g, or 0.1 mL/g to an upper limit of about 0.15 mL/g, 0.12 mL/g, or 0.1 mL/g, and wherein the pore volume may range from any lower limit to any upper limit and encompass any subset therebetween. As used herein, the term “pore volume” refers to the pore volume measured with BET analysis with nitrogen gas, which is sometimes referred to as the micropore volume. The term “pore volume” does not refer to the volume of the macropores measured by visualization, for example, with a scanning electron micrograph.

In some embodiments, the average pore size of the polysaccharide ester microspheres may range from a lower limit of about 20 nm or 25 nm to an upper limit of about 30 nm or 25 nm, and wherein the average pore size may range from any lower limit to any upper limit and encompass any subset therebetween. As used herein, the term “average pore size” refers to the pore volume measured with BET analysis with nitrogen gas, which is sometimes referred to as the average micropore size. The term “average pore size” does not refer to the size of the macropores measured by visualization, for example, with a scanning electron micrograph.

In some embodiments, the polysaccharide ester microspheres may have a crystallinity ranging from a lower limit of about 0%, 5%, or 10% to an upper limit of about 70%, 60%, 50%, or 40%, and wherein the crystallinity may range from any lower limit to any upper limit and encompass any subset therebetween. One suitable method for determining porosity is X-ray diffraction.

In some embodiments, the polysaccharide ester microspheres may have a crush strength ranging from a lower limit of about 5 g, 7 g, 10 g, or 15 g to an upper limit of about 45 g, 30 g, 25 g, 20 g, or 15 g as the mass required to produce a 10% loss in particle diameter, and wherein the crash strength may range from any lower limit to any upper limit and encompass any subset therebetween. One suitable method for determining crush strength is ASTM D 3102-72.

In some embodiments, the polysaccharide ester microspheres may have an average particle size ranging from a lower limit of about 100 nm, 250 nm, 500 nm, 1 micron, 5 microns, 25 microns, or 100 microns to an upper limit of about 2000 microns, 1000 microns, 750 microns, 500 microns, 250 microns, or 100 microns, and wherein the average particle size may range from any lower limit to any upper limit and encompass any subset therebetween. Suitable methods for determining average particle size include sieve techniques for larger particle sizes and laser diffraction analyzers for smaller particle sizes. As used herein, the term “average particle size” refers to the D₅₀ by weight (i.e., the diameter where 50% by weight of the microspheres have a lower diameter).

In some embodiments, the polysaccharide ester microspheres may have a dye uptake ranging from a lower limit of about 0.1 g, 0.5 g, or 1 g per 100 g of polysaccharide ester microspheres per 4 hours to an upper limit of about 5 g, 3 g, or 2 g per 100 g of polysaccharide ester microspheres per 4 hours as measured using Acid Blue 290, and wherein the dye uptake may range from any lower limit to any upper limit and encompass any subset therebetween.

In some embodiments, polysaccharide ester microspheres may optionally further comprise additives. Exemplary additives may include, but are not limited to, plasticizers, dyes, pigments, pore forming agents, active pharmaceuticals (e.g., hydrophilic active pharmaceutical, hydrophobic active pharmaceutical, amphoteric active pharmaceutical, pain relievers, antibiotics, steroids, and antioxidants), prodrugs of active pharmaceuticals, active biologicals (e.g., hormones, DNAs, RNAs, siRNAs, peptides, enzymes, nucleotides, oligionucleotides, antibodies, and monoclonal antibodies), antibiotics, antifungals, antitoxins, antigens, therapeutics (e.g., chemotherapeutics, radiation-poisoning therapeutics, radioisotopes), preventive therapeutics (e.g., antioxidants, radiation mitigation agents, and vaccines), nutritional supplements (e.g., vitamins, minerals, nutraceuticals, metabolism enhancing agents, and antioxidants), imaging agents (e.g., magnetic resonance imaging contrast agents, x-ray imaging contrast agents, and radioisotopes), food agents (e.g., preservatives, probiotics, enzymes, colors, pigments, sweeteners, water, flavorants, and aromas), flavorants, olfactory agents (e.g., fragrances and aromas), plant agents (e.g., agricultural active ingredients, pesticides, fertilizers, and hormones), chemical-reaction agents (e.g., chemical crosslinkers and catalysts), insect repellents, and the like, and any combination thereof. Nonlimiting examples of at least some of these additives are provided herein.

Additives may in some embodiments, be included in a polysaccharide ester dope described herein. In some embodiments, additives may be absorbed into and/or adsorb to polysaccharide ester microspheres after the forming process (1.6). Combinations of the aforementioned may be suitable in some embodiments. In continuous processes described herein, areas for the incorporation of additives may optionally be included.

Polysaccharide ester microspheres may have a combination of the properties described herein (e.g., crystallinity, crush strength, surface area, average particle size, particle size distribution, dye uptake, and the like) and comprise at least one polysaccharide ester described herein (e.g., based on molecular weight, organic ester substituent, degree of substitution, inorganic ester substituent and amount thereof, hydrophobicity, and the like) and optionally further comprise at least one additive described herein. Such polysaccharide ester microspheres may be formed by any suitable method described herein (e.g., batch, continuous, or hybrid methods in combination with methods for producing dopes that include or exclude finishing processes (or that produce dopes from multiple methods that independently include or exclude finishing processes)).

### III. Applications and Articles Comprising Polysaccharide Ester Microspheres

In some embodiments, polysaccharide ester microspheres described herein may be utilized for the controlled release of additives, e.g., active pharmaceuticals, prodrugs of active pharmaceuticals, active biologicals, antibiotics, antifungals, antitoxins, antigens, therapeutics, preventive therapeutics, nutritional supplements, imaging agents, food agents, flavorants, olfactory agents, plant agents, chemical-reaction agents, insect repellents, and the like, and any combination thereof.

In some embodiments, articles may comprise polysaccharide ester microspheres described herein. Exemplary examples of articles suitable for use in conjunction with polysaccharide ester microspheres may include, but are not limited to, pharmaceutical compositions (e.g., excipients and patches), cosmetics compositions (e.g., liquids, creams, lotions, sprays, lipstick, color cosmetics, and mascara), agricultural compositions (e.g., plant nutritional supplements and pesticide compositions), food products (e.g., flavorant enhancers, aroma enhancers, nutritional enhancers), ballistic applications such as explosives and propellants, filters (e.g., cigarette filters, air filters, and water filters), filter packing, chromatographic packing, specialty filters, any hybrid thereof, and the like.

By way of nonlimiting example, a pharmaceutical patch may, in some embodiments, comprise in order a backing layer, an active layer that comprises polysaccharide ester microspheres described herein, and an adhesive layer. The polysaccharide ester microspheres in this and other similar applications may, in some embodiments, advantageously comprise high concentrations of an additive to be released (e.g., an active pharmaceutical, a nutritional supplement, and the like described herein) and function as a reservoir for the additive. This may advantageously allow for higher doses and/or longer lifetime articles.

By way of another nonlimiting example, an oral pharmaceutical delivery vehicle (e.g., a tablet, a capsule, or a liquid-gel) may, in some embodiments, comprise polysaccharide ester microspheres described herein. The polysaccharide ester microspheres in this and other similar applications may, in some embodiments, advantageously comprise an additive to be released (e.g., an active pharmaceutical, a nutraceutical, and the like described herein) and function to control the release rate of the additive. In some embodiments, the delivery vehicle may function to control the release of the microcapsules, thereby enabling, in some embodiments, a higher degree of release timing and release rate of the additive.

By way of yet another nonlimiting example, an agricultural product (e.g., a spray, a concentrate, or the like) may comprise polysaccharide ester microspheres described herein dispersed in a fluid (e.g., water or an emulsion). The polysaccharide ester microspheres in this and other similar applications may, in some embodiments, advantageously comprise plant agents like herbicides, fungicides, insecticides, bactericides, nitrogen sources, growth promoters, and the like, and any combination thereof. In some embodiments, the polysaccharide ester microspheres may be designed so as to change the release rate with changes in pH. In some embodiments, the polysaccharide ester microspheres may be designed to have an increased release rate after a rain and/or watering, e.g., due to a concentration differential and the surrounding environment.

By way of another nonlimiting example, a food or beverage product may, in some embodiments, comprise polysaccharide ester microspheres described herein. The polysaccharide ester microspheres in this and other similar applications may, in some embodiments, comprise additives like flavorants, aromas, and/or colorants and serve to provide for a desired quality (e.g., color, flavor, and/or aroma intensity) of the food or beverage product over an extended time frame (e.g., to better match the preservative lifetime of the food or beverage product). The polysaccharide ester microspheres in this and other similar applications may, in some embodiments, comprise additives like flavorants, aromas, and/or colorants and serve to provide for release of a color, flavor, and/or aroma upon rupture (e.g., during chewing) (e.g., gums that change the color of the tongue or release different flavors when chewed, dissolvable strips, meal-replacement products, ice creams, and the like). The polysaccharide ester microspheres in this and other similar applications may, in some embodiments, comprise additives like vitamins, nutraceuticals, pharmaceutical, and the like and serve to enhance the nutritional value of or deliver pharmaceuticals via the food or beverage product (e.g., dissolvable strips, candy products, powdered beverage mixes, yogurts, granola bars, meal-replacement products, nicotine gums, and the like). In some embodiments, combinations of the foregoing additives and/or combinations of the foregoing polysaccharide ester microspheres may be suitable for use in conjunction with food or beverage products.

By way of yet another nonlimiting example, a food or beverage packaging may, in some embodiments, comprise polysaccharide ester microspheres described herein. The polysaccharide ester microspheres in this and other similar applications may, in some embodiments, comprise additives like aromas and be incorporated into the packaging so as to release an aroma burst upon opening the package (e.g., in an adhesive seal). The polysaccharide ester microspheres in this and other similar applications may, in some embodiments, comprise additives like flavorants and/or aromas and be incorporated into the edible packaging so as to release the additive upon chewing or dissolution of the packaging (e.g., edible gum wrappers).

By way of another nonlimiting example, a cosmetic (e.g., bronzers, face powder, eye shadow, eyeliner, mascara, blush, brow powder, baby powder, lip-gloss, lipstick, and the like) may, in some embodiments, comprise polysaccharide ester microspheres described herein. The polysaccharide ester microspheres in this and other similar applications may, in some embodiments, comprise additives like colorants, flavorants, aromas, deodorants, nutraceuticals, and the like. In some embodiments, the ability of polysaccharide ester microspheres to uptake and hold a high concentration of the colorant, which may be particularly advantageous in cosmetics with intense colors (e.g., eye shadows, blushes, bronzers, lip-glosses, lipsticks, and the like. Further, in some embodiments, it has been observed that polysaccharide ester microspheres described herein may provide a luster to the surface to which they are applied, which may be advantageous in cosmetic powder applications.

By way of yet another nonlimiting example, a lotion or cream may, in some embodiments, comprise polysaccharide ester microspheres described herein. The polysaccharide ester microspheres in this and other similar applications may, in some embodiments, comprise additives like colorants (e.g., for bronzing), aromas, deodorants, nutraceuticals, and the like. In some embodiments, polysaccharide ester microspheres suitable for use in conjunction with lotions may be smaller in size to minimize or eliminate an associated feel (e.g., about 50 microns or less) or may be larger (e.g., greater than about 50 microns) to elicit an abrasive feel (e.g., in exfoliating lotions or creams).

By way of another nonlimiting example, a hair product (e.g., shampoo, conditioner, oil, dye, and the like) may, in some embodiments, comprise polysaccharide ester microspheres described. The polysaccharide ester microspheres in this and other similar applications may, in some embodiments, comprise additives like colorants, aromas (e.g., essential oils), deodorants, moisturizers, nutraceuticals (e.g., vitamin E), and the like.

By way of another nonlimiting example, a deodorant (e.g., sprays, roll-on, powders, and the like) may, in some embodiments, comprise polysaccharide ester microspheres described herein. The polysaccharide ester microspheres in this and other similar applications may, in some embodiments, comprise additives like aromas (e.g., essential oils), deodorants, moisturizers, nutraceuticals (e.g., vitamin E), and the like. In some embodiments, the polysaccharide ester microspheres may be designed to be abrasion activated (e.g., increase release under mechanical pressure) so as to increase release of the additive when activity increases. In some embodiments, the polysaccharide ester microspheres may be designed to increase release based on pH so as to increase release of the additive when the local environment's pH changes (e.g., during sweating).

By way of yet another nonlimiting example, a cigarette filter may, in some embodiments, comprise polysaccharide ester microspheres described herein. In some instance, the cigarette filter may be a cellulose acetate tow filter, a paper filter, or the like with the polysaccharide ester microspheres dispersed throughout the filter, optionally adhered to the material of the filter (e.g., the cellulose acetate tow, the paper, or the like). In some instances, the cigarette filter may comprise a cavity and/or a capsule with the microspheres contained therein. In some instances, a hybrid of the foregoing may be useful. The polysaccharide ester microspheres in this and other similar applications may, in some embodiments, comprise additives like aromas, flavorants, and the like. In some embodiments, the act of drawing on the cigarette filter may decrease the air pressure about the polysaccharide ester microspheres, thereby increasing the release of the additive therein.

By way of another nonlimiting example, an explosive device and/or a propellant may, in some embodiments, comprise polysaccharide ester microspheres described herein. The polysaccharide ester microspheres in this and other similar applications may, in some embodiments, comprise additives like an explosive material, a fuel material, a fuel additive, and the like. In some embodiments, the polysaccharide ester microspheres in this and similar applications may be designed to increase release under mechanical pressure, which may act as a trigger for explosive device and/or propellant while enabling safe transport otherwise.

By way of yet another nonlimiting example, an adhesive may, in some embodiments, comprise polysaccharide ester microspheres described herein. In some embodiments, the polysaccharide ester microspheres may be utilized as a substitute for tackifiers and/or viscosity modifiers in adhesives and optionally comprise an additive useful in a desired application (e.g., a pharmaceutical for the adhesive in transdermal patch, an aroma in a food packaging adhesive, and the like).

In some embodiments, the polysaccharide ester microspheres described herein may be administered to a patient. As used herein, the term “subject” and “patient” are used interchangeably herein and refer to both human and nonhuman animals and insects. The term “nonhuman animals” as used herein includes all vertebrates, e.g., mammals and non-mammals, such as nonhuman primates, mice, rats, sheep, dogs, cats, horses, cows, chickens, amphibians, fish, reptiles, and the like. The term “insects” as used herein includes all arthropods, e.g., bees, flies, Drosophila flies, beetles, spiders, and the like.

In some embodiments, the polysaccharide ester microspheres described herein may be administered to patients orally (e.g., pills, tablets, and the like), subdermally (e.g., subdermal implants), transdermally (e.g., patches, lotions, cosmetics, and the like), transmucosally (e.g., oromucosal inserts, intrauterine devices, intravaginal rings, dental fibers, and the like), and/or as a part of an implantable medical device. In some embodiments, additives in polysaccharide ester microspheres may be administered to patients by oral delivery of the polysaccharide ester microspheres, subdermal implantation or injection of the polysaccharide ester microspheres, placement of the polysaccharide ester microspheres for transdermal administration of the additive, and/or implanting a medical device including polysaccharide ester microspheres described herein.

A typical dosage of an additive (e.g., active pharmaceuticals and prodrugs of active pharmaceuticals) might range from about 0.001 mg/kg to about 1000 mg/kg, preferably from about 0.01 mg/kg to about 100 mg/kg, and more preferably from about 0.10 mg/kg to about 20 mg/kg, relative to weight of the patient. In some embodiments, active pharmaceuticals and prodrugs of active pharmaceuticals may be used alone or in combination with other additives. One skilled in the art should understand the dose and/or combination of additives should be chosen so as to minimize adverse interactions.

In some embodiments, the polysaccharide ester microspheres described herein may be a component of a kit. In some embodiments, a kit may include a set of instructions and at least one type of polysaccharide ester microspheres. In some embodiments, a kit may include a set of instructions and an article comprising at least one type polysaccharide ester microsphere described herein.

### IV. Additives

Exemplary additives may include, but are not limited to, plasticizers, dyes, pigments, pore forming agents, active pharmaceuticals (e.g., hydrophilic active pharmaceutical, hydrophobic active pharmaceutical, amphoteric active pharmaceutical, pain relievers, antibiotics, steroids, and antioxidants), prodrugs of active pharmaceuticals, active biologicals (e.g., hormones, DNAs, RNAs, siRNAs, peptides, enzymes, nucleotides, oligionucleotides, antibodies, and monoclonal antibodies), antibiotics, antifungals, antitoxins, antigens, therapeutics (e.g., chemotherapeutics, radiation-poisoning therapeutics, radioisotopes), preventive therapeutics (e.g., antioxidants, radiation mitigation agents, and vaccines), nutritional supplements (e.g., vitamins, nutraceuticals, metabolism enhancing agents, and antioxidants), imaging agents (e.g., magnetic resonance imaging contrast agents, x-ray imaging contrast agents, and radioisotopes), food agents (e.g., preservatives, fragrances, and aromas), flavorants, olfactory agents (e.g., fragrances and aromas), plant agents (e.g., agricultural active ingredients, pesticides, fertilizers, and hormones), chemical-reaction agents (e.g., chemical crosslinkers and catalysts), insect repellents, and the like, and any combination thereof.

Examples of plasticizers may include, but are not limited to, water, glycerol triacetate (triacetin), diacetin, triethyl citrate, acetyl trimethyl citrate, dimethoxy-ethyl phthalate, dimethyl phthalate, diethyl phthalate, dibutyl phthalate, diaryl phthalate, methyl phthalyl ethyl glycolate, o-phenyl phenyl-(bis) phenyl phosphate, 1,4-butanediol diacetate, diacetate, dipropionate ester of triethylene glycol, dibutyrate ester of triethylene glycol, tributyl phosphate, glycerin, glycerin esters, diacetyl glycerin, monoacetyl glycerin, glycerol, polyethylene glycol, diethylene glycol, polypropylene glycol, polyglycoldiglycidyl ethers, dimethyl sulfoxide, alkylphosphate esters, polycaprolactone, di-2-methoxyethyl phthalate, dibutyl tartrate, ethyl o-benzoylbenzoate, ethyl phthalyl ethyl glycolate, methyl phthalyl ethyl glycolate, n-ethyltoluenesulfonamide, o-cresyl p-toluenesulfonate, trimethyl phosphate, triethyl phosphate, tributyl phosphate, triphenyl phosphate, tripropionin, polycaprolactone, and the like, any derivative thereof, and any combination thereof.

Examples of pigments and dyes may include, but are not limited to, titanium dioxide, silicon dioxide, tartrazine, E102, phthalocyanine blue, phthalocyanine green, quinacridones, perylene tetracarboxylic acid di-imides, dioxazines, perinones disazo pigments, anthraquinone pigments, carbon black, metal powders, iron oxide, ultramarine, calcium carbonate, kaolin clay, aluminum hydroxide, barium sulfate, zinc oxide, aluminum oxide, CARTASOL® dyes (cationic dyes, available from Clariant Services) in liquid and/or granular form (e.g., CARTASOL® Brilliant Yellow K-6G liquid, CARTASOL® Yellow K-4GL liquid, CARTASOL® Yellow K-GL liquid, CARTASOL® Orange K-3GL liquid, CARTASOL® Scarlet K-2GL liquid, CARTASOL® Red K-3BN liquid, CARTASOL® Blue K-5R liquid, CARTASOL® Blue K-RL liquid, CARTASOL® Turquoise K-RL liquid/granules, CARTASOL® Brown K-BL liquid), FASTUSOL® dyes (an auxochrome, available from BASF) (e.g., Yellow 3GL, Fastusol C Blue 74L), and the like, any derivative thereof, and any combination thereof.

Examples of pore forming agents may include, but are not limited to, polyethylene glycol, triacetin, dimethyl phthalate, ethylene diacetate, sorbitol, magnesium sulfate, and the like, and any combination thereof.

Examples of suitable agents (active agents (e.g., active pharmaceuticals and prodrugs of active pharmaceuticals), removal agents, and tracking agents) may include, but are not limited to, 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, abamectin, abanoquil, abcizimab (commercially available as REOPRO® from Eli Lilly and Company), abecarnil, abiraterone, ablukast, ablukast sodium, acadesine, acamprosate, acarbose, acebutolol, acecainide hydrochloride, aceclidine, aceclofenae, acedapsone, aceglutamide aluminum, acemannan, acetaminophen, acetazolamide, acetohexamide, acetohydroxamic acid, acetomepregenol, acetophenazine maleate, acetosulfone sodium, acetylcholine chloride, acetylcysteine, acetyl-L-carnitine, acetylmethadol, acifran, acipimox, acitemate, acitretin, acivicin, aclarubicin, aclatonium, acodazole hydrochloride, aconiazide, acrisorcin, acrivastine, acronine, actisomide, actodigin, acyclovir, acylfulvene, adafenoxate, adalimumab (commercially available as HUMIRA® from Abbott Laboratories), adapalene, adapalene, adatanserin, adatanserin hydrochloride, adecypenol, adecypenol, adefovir, adelmidrol, ademetionine, adenosine, adinazolam, adipheinine hydrochloride, adiposin, adozelesin, adrafinil, adrenalone, airbutamine, alacepril, alamecin, alanine, alaproclate, alaptide, albendazole, albolabrin, albuterol (commercially available as VENTOLIN® from GlaxoSmithKline), albutoin, alclofenae, alclometasone dipropionate, aluminum chlorhydroxyallantoinate (commercially available as ALCOLOXA® from TRI-K Industries, Inc.), aldecalmycin, aldesleukin, aldioxa, alendronate sodium (commercially available as FOSAMAX® from Merck), alendronic acid, alentemol, alentemol hydrobromide, aletamine hydrochloride, aleuronium chloride, alexidine, alfacalcidol, alfentanil hydrochloride, alfuzosin, algestone acetonide, alglucerase, aliflurane, alinastine, alipamide, allantoin, allobarbital, allopurinol, a tachy-kinins (TK) antagonist, alonimid, alosetron, alosetron hydrochloride, alovudine, alpertine, alpha amylase, alpha idosone, alpidem, alprazolam (commercially available as XANAX® from Pfizer, Inc.), alprenolol hydrochloride, alprenoxime hydrochloride, alprostadil, alrestatin sodium, altanserin tartrate, alteplase, althiazide, altretamine, altromycin B, alverinc citrate, alvircept sudotox, amadinone acetate, amantadine hydrochloride, ambamustine, ambomycin, ambruticin, ambuphylline, ambuside, amcinafal, amcinonide, amdinocillin, amdinocillin pivoxil, amedalin hydrochloride, amelometasone, ameltolide, amesergide, ametantrone acetate, amezinium metilsulfate, amfebutamone, amfenac sodium, amflutizole, amicycline, amidephrine mesylate, amidox, amifloxacin, amifostine, amikacin, amiloride hydrochloride, aminacrine hydrochloride, aminobenzoate potassium, aminobenzoate sodium, aminocaproic acid, aminoglutethimide, aminohippurate sodium, aminolevulinic acid, aminophylline, aminorex, aminosalicylate sodium, aminosalicylic acid, amiodarone, amiprilose hydrochloride, amiquinsin hydrochloride, amisulpride, amitraz, amitriptyline hydrochloride, amlexanox, amlodipine, amobarbital sodium, amodiaquine, amodiaquine hydrochloride, amorolfine, amoxapine, amoxicillin, amphecloral, amphetamine sulfate, amphomycin, amphotericin B, ampicillin, ampiroxicam, ampyzine sulfate, amquinate, amrinone, aminone, amrubicin, amsacrine, amythiamicin, anagestone acetate, anagrelide, anakinra, ananain, anaritide, anaritide acetate, anastrozole (commercially available as ARIMIDEX® from AstraZeneca), anazolene sodium, ancrod, andrographolide, androstenedione, angiogenesis inhibitors, angiotensin amide, anidoxime, anileridine, anilopam hydrochloride, aniracetam, anirolac, anisotropine methylbromide, anistreplase, anitrazafen, anordrin, antagonist D, antagonist G, antarelix, antazoline phosphate, anthelmycin, anthralin, anthramycin, antiandrogen, antihemophilic factor (commercially available as XYNTHA® from Pfizer, Inc.), acedapsone, felbamate, antiestrogen, antineoplaston, antipyrine, antisense oligonucleotides, apadoline, apafant, apalcillin sodium, apaxifylline, apazone, aphidicolin glycinate, apixifylline, apomorphine hydrochloride, apraclonidine, apraclonidine hydrochloride, apramycin, aprindine, aprindine hydrochloride, aprosulate sodium, aprotinin, aptazapine maleate, aptiganel, apurinic acid, apurinic acid, aranidipine, aranotin, arbaprostil, arbekicin, 1-methyl-2-((phenylthio)methyl)-3-carbethoxy-4-((dimethylamino)methyl)-5-hydroxy-6-bromindole (commercially available as ARBIDOL® from Masterlek), arbutamine hydrochloride, arclofenin, ardeparin sodium, (2R,4R)-1-[(2S)-5-(diaminomethylideneamino)-2-[[(3R)-3-methyl-1,2,3,4-tetrahydroquinolin-8-yl]sulfonylamino]pentanoyl]-4-methyl-piperidine-2-carboxylic acid (commercially available as ARGATROBAN® from GlaxoSmithKline), arginine, argipressin tannate, arildone, aripiprazol, arotinolol, arpinocid, arteflene, artilide fumarate, asimadoline, aspalatone, asparaginase, aspartic acid, aspartocin, asperfuran, aspirin, aspoxicillin, asprelin, astemizole, astromicin sulfate, asulacrine, atamestane, atenolol, atevirdine, atipamezole, atiprosin maleate, atolide, atorvastatin (commercially available as LIPITOR® from Pfizer, Inc.), atosiban, atovaquone, atpenin B, atracurium besylate, atrimustine, atrinositol, atropine, auranofin, aureobasidin A, aurothioglucose, avilamycin, avoparcin, avridine, nizatidine (commercially available as AXID® from GlaxoSmithKline), axinastatin 1, axinastatin 2, axinastatin 3, azabon, azacitidinie, azaclorzine hydrochloride, azaconazole, azadirachtine, azalanstat dihydrochloride, azaloxan fumarate, azanator maleate, azanidazole, azaperone, azaribine, azaserine, azasetron, azatadine maleate, azathioprine, azathioprine sodium, azatoxin, azatyrosine, azelaic acid, azelastine, azelnidipine, azepindole, azetepa, azimilide, azithromycin, azlocillin, azolimine, azosemide, azotomycin, aztreonam, azumolene sodium, bacampicillin hydrochloride, baccatin III, bacitracin, baclofen, bacoside A, bacoside B, bactobolamine, balanol, balazipone, balhimycin, balofloxacin, balsalazide, bambermycins, bambuterol, bamethan sulfate, bamifylline hydrochloride, bamidazole, baohuoside 1, barmastine, barnidipine, basifungin, batanopride hydrochloride, batebulast, batelapine maleate, batimastat, beauvericin, becanthone hydrochloride, becaplermin, becliconazole, beclomethasone dipropionate, befloxatone, beinserazide, belfosdil, belladonna, beloxamide, bemesetron, bemitradine, bemoradan, benapryzine hydrochloride, benazepril hydrochloride, benazeprilat, bendacalol mesylate, bendazac, bendroflumethiazide, benflumetol, benidipine, benorterone, benoxaprofen, benoxaprofen, benoxinate hydrochloride, benperidol, bentazepam, bentiromide, benurestat, benzbromarone, benzethonium chloride, benzetimide hydrochloride, benzilonium bromide, benzindopyrine hydrochloride, benzisoxazole, benzocaine, benzochlorins, benzoctamine hydrochloride, benzodepa, benzoidazoxan, benzonatate, benzoyl peroxide, benzoylpas calcium, benzoylstaurosporine, benzquinamide, benzthiazide, benztropine, benztropine mesylate, benzydamine hydrochloride, benzylpenicilloyl polylysine, bepridil, bepridil hydrochloride, beractant, beraprost, berefrine, berlafenone, bertosamil, berythromycin, besipirdine, beta-alethine, betaclamycin B, betamethasone, betamipron, betaxolol, betaxolol hydrochloride, bethanechol chloride, bethanidine sulfate, betulinic acid, bevacizumab (commercially available as AVASTIN® available from Genenetech), bevantolol, bevantolol hydrochloride, bezafibrate, bFGF inhibitor, bialamicol hydrochloride, biapenem, bicalutamide, bicifadine hydrochloride, biclodil hydrochloride, bidisomide, bifemelane, bifonazole, bimakalim, bimithil, bindarit, biniramycin, binospirone, bioxalomycin alpha2, bipenamol hydrochloride, biperiden, biphenamine hydrochloride, biriperone, bisantrene, bisaramil, bisaziridinylspermine, bis-benzimidazole A, bis-benzimidazole B, bisnafide, bisobrin lactate, bisoprolol, bispyrithione magsulfex, bistramide D, bistramide K, bistratene A, bithionolate sodium, bitolterol besylate, bivalirudin, bizelesin, bleomycin sulfate, bolandiol dipropionate, bolasterone, boldenone undecylenate, boldine, bolenol, bolmantalate, bopindolol, bosentan, boxidine, brefeldin, breflate, brequinar sodium, bretazenil, bretylium bosylate, brifentanil hydrochloride, brimonidine, brinolase, brocresine, brocrinat, brofoxine, bromadoline maleate, bromazepam, bromchlorenone, bromelains, bromfenac, brominidione, bromocriptine, bromodiphenhydramine hydrochloride, bromoxamide, bromperidol, bromperidol decanoate, brompheniramine baleate, broperamole, bropirimine, brotizolam, bucamide maleate, bucindolol, buclizine hydrochloride, bucromarone, budesonide (commercially available as RHINOCORT® and ENTOCORT® from AstraZeneca), budipine, budotitane, buformin, bumetanide, bunaprolast, bunazosin, bunolol hydrochloride, bupicomide, bupivacaine hydrochloride, buprenorphine hydrochloride, bupropion hydrochloride, buramate, buserelin acetate, buspirone hydrochloride, busulfan, butabarbital, butacetin, butaclamol hydrochloride, butalbital, butamben, butamirate citrate, butaperazine, butaprost, butedronate tetrasodium, butenafine, buterizine, buthionine sulfoximine, butikacin, butilfenin, butirosin sulfate, butixirate, butixocort propionate, butoconazole nitrate, butonate, butopamine, butoprozine hydrochloride, butorphanol, butoxamine hydrochloride, butriptyline hydrochloride, cactinomycin, cadexomer iodine, caffeine, calanolide A, calcifediol, calcipotriene, calcipotriol, calcitonin, calcitriol, calcium undecylenate, calphostin C, calusterone, cambendazole, camonagrel, camptothecin derivatives, canarypox IL-2, candesartan, candicidin, candoxatril, candoxatrilat, caniglibose, canrenoate potassium, canrenone, capecitabine, capobenate sodium, capobenic acid, capreomycin sulfate, capromab, capsaicin, captopril, capuride, caracemide, carbachol, carbadox, carbamazepine, carbamide peroxide, carbantel lauryl sulfate, carbaspirin calcium, carbazeran, carbazomycin C, carbenicillin potassium, carbenoxolone sodium, carbetimer, carbetocin, carbidopa, carbidopa-levodopa, carbinoxamine maleate, carbiphene hydrochloride, carbocloral, carbocysteine, carbol-fuchsin, carboplatin, carboprost, carbovir, carboxamide-amino-triazole, carboxyamidotriazole, carboxymethylated beta-1,3-glucan, carbuterol hydrochloride, CaRest M3, carfentanil citrate, carisoprodol, carmantadine, carmustine, CARN 700, camidazole, caroxazone, carperitide, carphenazine maleate, carprofen, carsatrin succinate, cartazolate, carteolol, carteolol hydrochloride, cartilage derived inhibitor, carubicin hydrochloride, carumonam sodium, carvedilol, carvotroline, carvotroline hydrochloride, carzelesin, casein kinase inhibitors (ICOS), castanospermine, caurumonam, cebaracetam, cecropin B, cedefingol, cefaclor, cefadroxil, cefamandole, cefaparole, cefatrizine, cefazaflur sodium, cefazolin, cefbuperazone, cefcapene pivoxil, cefdaloxime pentexil tosilate, cefdinir, cefditoren pivoxil, cefepime, cefetamet, cefetecol, cefixime, cefluprenam, cefinenoxime hydrochloride, cefinetazole, cefminlox, cefodizime, cefonicid sodium, cefoperazone sodium, ceforanide, cefoselis, cefotaxime sodium, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftibuten, ceftizoxime sodium, ceftriaxone, cefuroxime, celastrol, celikalim, celiprolol, cepacidiine A, cephacetrile sodium, cephalexin, cephaloglycin, cephaloridine, cephalothin sodium, cephapirin sodium, cephradine, cericlamine, cerivastatin, ceronapril, certoparin sodium, ceruletide, cetaben sodium, cetalkonium chloride, cetamolol hydrochloride, cetiedil, cetirizine, cetophenicol, cetraxate hydrochloride, cetrorelix, cetuximab (commercially available as ERBITUX® from Eli Lilly and Company), cetylpyridinium chloride, chenodiol, chlophedianol hydrochloride, chloral betaine, chlorambucil, chloramphenicol, chlordantoin, chlordiazepoxide, chlorhexidine gluconate, chlorins, chlormadinone acetate, chloroorienticin A, chloroprocaine hydrochloride, chloropropamide, chloroquine, chloroquinoxaline sulfonamide, chlorothiazide, chlorotrianisene, chloroxine, chloroxylenol, chlorphenesin carbamate, chlorpheniramine maleate, chlorpromazine, chlorpropamide, chlorprothixene, chlortetracycline bisulfate, chlorthalidone, chlorzoxazone, cholestyramine resin, chromonar hydrochloride, cibenzoline, cicaprost, ciclafrine hydrochloride, ciclazindol, ciclesonide, cicletanine, ciclopirox, cicloprofen, cicloprolol, cidofovir, cidoxepin hydrochloride, cifenline, ciglitazone, ciladopa hydrochloride, cilansetron, cilastatin sodium, cilazapril, cilnidipine, cilobamine mesylate, cilobradine, cilofungin, cilostazol, cimaterol, cimetidine, cimetropium bromide, cinalukast, cinanserin hydrochloride, cinepazet maleate, cinflumide, cingestol, cinitapride, cinnamedrine, cinnarizine, cinolazepam, cinoxacin, cinperene, cinromide, cintazone, cintriamide, cioteronel, cipamfylline, ciprefadol succinate, ciprocinonide, ciprofibrate, ciprofloxacin, ciprostene, ciramadol, cirolemycin, cisapride, cisatracurium besilate, cisconazole, cisplatin, cis-porphyrin, cistinexine, citalopram, citenamide, citicoline, citreamicin alpha, cladribine, clamoxyquin hydrochloride, clarithromycin, clausenamide, clavulanate potassium, clazolam, clazolimine, clebopride, clemastine, Clentiazem maleate, clidinium bromide, clinafloxacin, clindamycin, clioquinol, clioxanide, cliprofen, clobazam, clobetasol propionate, clobetasone butyrate, clocortolone acetate, clodanolene, clodazon hydrochloride, clodronic acid, clof azimine, clofibrate, clofilium phosphate, clogestone acetate, clomacran phosphate, clomegestone acetate, clometherone, clomethiazole, clomifene analogues, clominorex, clomiphene, clomipramine hydrochloride, clonazepam, clonidine, clonitrate, clonixeril, clonixin, clopamide, clopenthixol, cloperidone hydrochloride, clopidogrel (commercially available as PLAVIX® from Bristol-Myers Squibb and Sanofi Pharmaceuticals), clopimozide, clopipazan mesylate, clopirac, cloprednol, cloprostenol sodium, clorazepate dipotassium, clorethate, clorexolone, cloroperone hydrochloride, clorprenaline hydrochloride, clorsulon, clortermine hydrochloride, closantel, closiramine aceturate, clothiapine, clothixamide maleate cloticasone propionate, clotrimazole, cloxacillin benzathine, cloxyquin, clozapine, cocaine, coccidioidin, codeine, codoxime, colchicine, colestimide, colestipol hydrochloride, colestolone, colforsin, colfosceril palmitate, colistimethate sodium, colistin sulfate, collismycin A, collismycin B, colterol mesylate, combretastatin A4, combretastatin analogue, complestatin, conagenin, conorphone hydrochloride, contignasterol, contortrostatin, cormethasone acetate, corticorelin ovine triflutate, corticotropin, cortisone acetate, cortivazol, cortodoxone, cosalane, costatolide, cosyntropin, cotinine, warfarin (commercially available as COUMADIN® from Bristol-Myers Squibb), coumermycin, crambescidin 816, crilvastatin, crisnatol, cromitrile sodium, cromolyn sodium, crotamiton, cryptophycin 8, cucumariosid, cuprimyxin, curacin A, curdlan sulfate, zinc hyaluran (commercially available as CURIOSIN® from Gedeon Richter), cyclacillin, cyclazocine, cyclazosin, cyclic HPMPC, cyclindole, cycliramine maleate, cyclizine, cyclobendazole, cyclobenzaprine, cyclobut A, cyclobut G, cyclocapron, cycloguanil pamoate, cycloheximide, cyclopentanthraquinones, cyclopenthiazide, cyclopentolate hydrochloride, cyclophenazine hydrochloride, cyclophosphamide, cycloplatam, cyclopropane, cycloserine, cyclosin, cyclosporine, cyclothialidine, cyclothiazide, cyclothiazomycin, cyheptamide, cypemycin, cypenamine hydrochloride, cyprazepam, cyproheptadine hydrochloride, cyprolidol hydrochloride, cyproterone, cyproximide, cysteamine, cysteine hydrochloride, cystine, cytarabine, cytarabine hydrochloride, cytarabine ocfosfate, cytochalasin B, cytolytic factor, cytostatin, dacarbazine, dacliximab, dactimicin, dactinomycin, daidzein, daledalin tosylate, dalfopristin, dalteparin sodium, daltroban, dalvastatin, danaparoid, danazol, dantrolene, daphlnodorin A, dapiprazole, dapitant, dapoxetine hydrochloride, dapsone, daptomycin, darglitazone sodium, darifenacin, darlucin A, darodipine, darsidomine, darusentan, daunorubicin hydrochloride, dazadrol maleate, dazepinil hydrochloride, dazmegrel, dazopride fumarate, dazoxiben hydrochloride, debrisoquin sulfate, decitabine, deferiprone, deflazacort, dehydrocholic acid, dehydrodidemnin B, dehydroepiandrosterone, delapril, delapril hydrochloride, delavirdine mesylate, delequamine, delfaprazine, delmadinone acetate, delmopinol, delphinidin, demecarium bromide, demeclocycline, demecycline, demoxepam, denofungin, deoxypyridinoline, 2-propylpentanoic acid (commercially available as DEPAKOTE® from Abbott), deprodone, deprostil, depsidomycin, deramciclane, dermatan sulfate, desciclovir, descinolone acetonide, desflurane, desipramine hydrochloride, desirudin, deslanoside, deslorelin, desmopressin, desogestrel, desonide, desoximetasone, desoxoamiodarone, desoxycorticosterone acetate, detajmium bitartrate, deterenol hydrochloride, detirelix acetate, devazepide, dexamethasone, dexamisole, dexbrompheniramine maleate, dexchlorpheniramine maleate, dexclamol hydrochloride, dexetimide, dexfenfluramine hydrochloride, dexifosfamide, deximafen, dexivacaine, dexketoprofen, dexloxiglumide, dexmedetomidine, dexormaplatin, dexoxadrol hydrochloride, dexpanthenol, dexpemedolac, dexpropranolol hydrochloride, dexrazoxane, dexsotalol, dextrin 2-sulphate, dextroamphetamine, dextromethorphan, dextrorphan hydrochloride, dextrothyroxine sodium, dexverapamil, dezaguanine, dezinamide, dezocine, diacetolol hydrochloride, diamocaine cyclamate, diapamide, diatrizoate meglumine, diatrizoic acid, diaveridine, diazepam, diaziquone, diazoxide, dibenzepin hydrochloride, dibenzothiophene, dibucaine, dichliorvos, dichloralphenazone, dichlorphenamide, dicirenone, diclofenac sodium, dicloxacillin, dicranin, dicumarol, dicyclomine hydrochloride, didanosine, didemnin B, didox, dienestrol, dienogest, diethylcarbamazine citrate, diethylhomospermine, diethylnorspermine, diethylpropion hydrochloride, diethylstilbestrol, difenoximide hydrochloride, difenoxin, diflorasone diacetate, difloxacin hydrochloride, difluanine hydrochloride, diflucortolone, diflumidone sodium, diflunisal, difluprednate, diftalone, digitalis, digitoxin, digoxin, dihexyverine hydrochloride, dihydrexidine, dihydro-5-azacytidine, dihydrocodeine bitartrate, dihydroergotamine mesylate, hihydroestosterone, dihydrostreptomycin sulfate, dihydrotachysterol, dihydrotaxol, phenytoin (commercially available as DILANTIN® from Parke, Davis & Company), dilevalol hydrochloride, diltiazem hydrochloride, dimefadane, dimefline hydrochloride, dimenhydrinate, dimercaprol, dimethadione, dimethindene maleate, dimethisterone, dimethyl prostaglandin A1, dimethyl sulfoxide, dimethylhomospermine, dimiracetam, dimoxamine hydrochloride, dinoprost, dinoprostone, dioxadrol hydrochloride, dioxamycin, diphenhydramine citrate, diphenidol, diphenoxylate hydrochloride, diphenyl spiromustine, dipivefin hydrochloride, dipivefrin, dipliencyprone, diprafenone, dipropylnorspermine, dipyridamole, dipyrithione, dipyrone, dirithromycin, discodermolide, disobutamide, disofenin, disopyramide, disoxaril, disulfuram, ditekiren, divalproex sodium, dizocilpine maleate, dobutamine, docarpamine, docebenone, docetaxel, doconazole, docosanol, dofetilide, dolasetron, drotrecogin alfa (commercially available as XIGRIS® from Eli Lilly and Company), duloxetine hydrochloride (commercially available as CYMBALTA® from Eli Lilly and Company), ebastine, ebiratide, ebrotidine, ebselen, ecabapide, ecabet, ecadotril, ecdisteron, echicetin, echistatin, echothiophate iodide, eclanamine maleate, eclazolast, ecomustine, econazole, ecteinascidin 722, edaravone, edatrexate, edelfosine, edifolone acetate, edobacomab, edoxudine, edrecolomab, edrophonium chloride, edroxyprogesteone acetate, efegatran, eflornithine, efonidipine, egualcen, elantrine, eleatonin, elemene, eletriptan, elgodipine, eliprodil, elsamitrucin, eltenae, elucaine, emalkalim, emedastine, emetine hydrochloride, emiglitate, emilium tosylate, emitefur, emoctakin, enadoline hydrochloride, enalapril, enalaprilat, enalkiren, enazadrem, encyprate, endralazine mesylate, endrysone, enflurane, englitazone, enilconazole, enisoprost, enlimomab, enloplatin, enofelast, enolicam sodium, enoxacin, enoxacin, enoxaparin sodium, enoxaparin sodium, enoximone, enpiroline phosphate, enprofylline, enpromate, entacapone, enterostatin, enviradene, enviroxime, ephedrine, epicillin, epimestrol, epinephrine, epinephryl borate, epipropidine, epirizole, epirubicin, epitetracycline hydrochloride, epithiazide, epoetin alfa, epoetin beta, epoprostenol, epoprostenol sodium, epoxymexrenone, epristeride, eprosartan, eptastigmine, equilenin, equilin, erbulozole, erdosteine, ergoloid mesylates, ergonovine maleate, ergotamine tartrate, ersentilide, ersofermin, erythritol, erythrityl tetranitrate, erythromycin, esmolol hydrochloride, esomeprazole (commercially available as NEXIUM® from AstraZeneca), esorubicin hydrochloride, esproquin hydrochloride, estazolam, estradiol, estramustine, estramustine analogue, estrazinol hydrobromide, estriol, estrofurate, estrogen agonists, estrogen antagonists, estrogens, conjugated estrogens, esterified, estrone, estropipate, esuprone, etafedrine hydrochloride, etanidazole, etanterol, etarotene, etazolate hydrochloride, eterobarb, ethacizin, ethacrynate sodium, ethacrynic acid, ethambutol hydrochloride, ethamivan, ethanolamine oleate, ethehlorvynol, ether, ethinyl estradiol, ethiodized oil, ethionamide, ethonam nitrate, ethopropazine hydrochloride, ethosuximide, ethotoin, ethoxazene hydrochloride, ethybenztropine, ethyl chloride, ethyl dibunate, ethylestrenol, ethyndiol, ethynerone, ethynodiol diacetate, etibendazole, etidocaine, etidronate disodium, etidronic acid, etifenin, etintidine hydrochloride, etizolam, etodolac, etofenamate, etoformin hydrochloride, etomidate, etonogestrel, etoperidone hydrochloride, etoposide, etoprine, etoxadrol hydrochloride, etozolin, etrabamine, etretinate, etryptamine acetate, eucatropine hydrochloride, eugenol, euprocin hydrochloride, eveminomicin, exametazine, examorelin, exaprolol hydrochloride, exemestane, exetimibe (commercially available as ZETIA® from Merck), fadrozole, faeriefungin, famciclovir, famotidine (commercially available as PEPCID® from Merck), fampridine, fantof arone, fantridone hydrochloride, faropenem, fasidotril, fasudil, fazarabine, fedotozine, felbamate, felbinac, felodipine, felypressin, fenalamide, fenamole, fenbendazole, fenbufen, fencibutirol, fenclofenac, fenclonine, fenclorac, fendosal, fenestrel, fenethylline hydrochloride, fenfluramine hydrochloride, fengabine, fenimide, fenisorex, fenmetozole hydrochloride, fenmetramide, fenobam, fenoctimine sulfate, fenofibrate, fenoldopam, fenoprofen, fenoterol, fenpipalone, fenprinast hydrochloride, fenprostalene, fenquizone, fenretinide, fenspiride, fentanyl citrate, fentiazac, fenticlor, fenticonazole, fenyripol hydrochloride, fepradinol, ferpifosate sodium, ferristene, ferrixan, ferrous sulfate, ferumoxides, ferumoxsil, fetoxylate hydrochloride, fexofenadine, fezolamine fumarate, fiacitabine, fialuridine, fibrinogen I 125, filgrastim, filipin, finasteride (commercially available as PROPECIA® from Merck), flavodilol maleate, flavopiridol, flavoxate hydrochloride, flazalone, flecamide, flerobuterol, fleroxacin, flesinoxan, flestolol sulfate, fletazepam, flezelastine, flobufen, floctafenine, flomoxef, flordipine, florfenicol, florifenine, flosatidil, flosequinan, floxacillin, floxuridine, fluasterone, fluazacort, flubanilate hydrochloride, flubendazole, flucindole, flucloronide, fluconazole, flucytosine, fludalanine, fludarabine phosphate, fludazonium chloride, fludeoxyglucose F 18, fludorex, fludrocortisone acetate, flufenamic acid, flufenisal, flumazenil, flumecinol, flumequine, flumeridone, flumethasone, flumetramide, flumezapine, fluminorex, flumizole, flumoxonide, flunarizine, flunidazole, flunisolide, flunitrazepam, flunixin, fluocalcitriol, fluocinolone acetonide, fluocinonide, fluocortin butyl, fluocortolone, fluorescein, fluorodaunorunicin hydrochloride, fluorodopa F 18, fluoroformylone, fluoroquinolones, fluorometholone, fluorouracil, fluotracen hydrochloride, fluoxetine, fluoxymesterone, fluparoxan, fluperamide, fluperolone acetate, fluphenazine decanoate, flupirtine, fluprednisolone, fluproquazone, fluprostenol sodium, fluquazone, fluradoline hydrochloride, flurandrenolide, flurazepam hydrochloride, flurbiprofen, fluretofen, flurithromycin, fluorocitabine, fluorof amide, fluorogestone acetate, flurothyl, fluoroxene, fluspiperone, fluspirilene, fluticasone propionate (commercially available as ADVAIR® from GlaxoSmithKline), fluticasone furoate, flutrimazole, flutroline, fluvastatin, fluvastatin sodium, fluvoxamine, fluzinamide, folic acid, follicle regulatory protein, folliculostatin, fomepizole, fonazine mesylate, forasartan, forfenimex, forfenirmex, formestane, formocortal, formoterol, fosarilate, fosazepam, foscarnet sodium, fosfomycin, fosfonet sodium, fosinopril, fosinoprilat, fosphenyloin, fosquidone, fostedil, fostriecin, fotemustine, fuchsin, basic, fumoxicillin, fungimycin, furaprofen, furazolidone, furazolium chloride, furegrelate sodium, furobufen, furodazole, furosemide, fusidate sodium, fusidic acid, gabapentin, gadobenate dimeglumine, gadobenic acid, gadobutrol, gadodiamide, gadolinium texaphyrin, gadopentetate dimegiumine, gadoteric acid, gadoteridol, gadoversetamide, galantamine, galdansetron, galdansetron hydrochloride, gallamine triethiodide, gallium nitrate, gallopamil, galocitabine, gamfexine, gamolenic acid, ganciclovir, ganirelix, ganirelix acetate, gelatinase inhibitors, gemcadiol, gemcitabine (commercially available as GEMZAR® from Eli Lilly and Company), gemeprost, gemfibrozil, gentamicin sulfate, gentian violet, gepirone, gestaclone, gestodene, gestonorone caproate, gestrinone, gevotroline hydrochloride, girisopam, glaspimod, glaucocalyxin A, glemanserin, gliamilide, glibornuride, glicetanile sodium, gliflumide, glimepiride, glipizide, gloximonam, glucagon, glutapyrone, glutathione inhibitors, glutethimide, glyburide, glycopine, glycopril, glycopyrrolate, glyhexamide, glymidine sodium, glyoctamide, glyparamide, colloidal gold Au 198, gonadoctrinins, gonadorelin, gonadotropins, goserelin, gramicidin, granisetron, grepafloxacin, griseofulvin, guaiapate, guaithylline, guanabenz, guanabenz acetate, guanadrel sulfate, guancydine, guanethidine monosulfate, guanfacine hydrochloride, guanisoquin sulfate, guanoclor sulfate, guanoctine hydrochloride, guanoxabenz, guanoxan sulfate, guanoxyfen sulfate, gusperimus trihydrochloride, halazepam, halcinonide, halichondrin B, halobetasol propionate, halof antrine, halof antrine hydrochloride, halofenate, halofuginone hydrobromide, halomon, galopemide, galoperidol, halopredone, haloprogesterone, haloprogin, halothane, halquinols, hamycin, han menopausal gonadotropins, hatomamicin, hatomarubigin A, hatomarubigin B, hatomarubigin C, hatomarubigin D, heparin sodium, hepsulfam, heregulin, hetacillin, heteronium bromide, hexachlorophene:hydrogen peroxide, hexafluorenium bromide, hexamethylene bisacetamide, hexedine, hexobendine, hexoprenaline sulfate, hexylresorcinol, histamine phosphate, histidine, histoplasmin, histrelin, homatropine hydrobromide, hoquizil hydrochloride, human chorionic gonadotropin, hycanthone, hydralazine hydrochloride, hydralazine polistirex, hydrochlorothiazide, hydrocodone bitartrate, hydrocortisone, hydroflumethiazide, hydromorphone hydrochloride, hydroxyamphetamine hydrobromide, hydroxychloroquine sulfate, hydroxyphenamate, hydroxyprogesterone caproate, hydroxyurca, hydroxyzine hydrochloride, hymecromone, hyoscyamine, hypericin, ibafloxacin, ibandronic acid, ibogaine, ibopamine, ibudilast, ibufenac, ibuprofen, ibutilide fumarate, icatibant acetate, ichthammol, icotidine, idarubicin, idoxifene, idoxuridine, idramantone, iemefloxacin, iesopitron, ifetroban, ifosfamide, ilepeimide, illimaquinone, ilmofosine, ilomastat, ilonidap, iloperidone, iloprost, imafen hydrochloride, imazodan hydrochloride, imidapril, imidazenil, imidazoacridones, imidecyl iodine, imidocarb hydrochloride, imidoline hydrochloride, imidurea, imiloxan hydrochloride, imipenem, imipramine hydrochloride, imiquimod, immunostimulant peptides, impromidine hydrochloride, indacrinone, indapamide, indecainide hydrochloride, indeloxazine hydrochloride, indigotindisulfonate sodium, indinavir, indocyanine green, indolapril hydrochloride, indolidan, indometacin, indomethacin sodium, indoprofen, indoramin, indorenate hydrochloride, indoxole, indriline hydrochloride, infliximab (commercially available as REMICADE® from Janssen Biotech, Inc.), inocoterone, inogatran, inolimomab, inositol niacinate, insulin, insulin glargine (commercially available as LANTUS® from Sanofi-Aventis), interferons, interferon beta-1a (commercially available as AVONEX® from BIOGEN), interleukins, intrazole, intriptyline hydrochloride, iobenguane, iobenzamic acid, iobitridol, iocarmate meglumine, iocarmic acid, iocetamic acid, iodamide, iodine, iodipamide meglumine, iodixanol, iodoamiloride, iodoantipyrine I 131, iodocholesterol I 131, iododoxorubicin, iodohippurate sodium I 131, iodopyracet I 125, iodoquinol, iodoxamate meglumine, iodoxamie acid, ioglicic acid, iofetamine hydrochloride I 123, iofratol, ioglucol, ioglucomide, ioglycamic acid, iogulamide, iohexyl, iomeprol, iomethin I 125, iopamidol, iopanoic acid, iopentol, iophendylate, ioprocemic acid, iopromide, iopronic acid, iopydol, iopydone, iopyrol, iosefamic acid, ioseric acid, iosulamide meglumine, iosumetic acid, iotasul, iotetric acid, iothalamate sodium, iothalamic acid, iotriside, iotrolan, iotroxic acid, iotyrosine I 131, ioversol, ioxagiate sodium, ioxaglate meglumine, ioxaglic acid, ioxilan, ioxotrizoic acid, ipazilide, ipenoxazone, ipidacrine, ipodate calcium, ipomeanol, 4-, ipratropium bromide, ipriflavone, iprindole, iprofenin, ipronidazole, iproplatin, iproxamine hydrochloride, ipsapirone, irbesartan, irinotecan, irloxacin, iroplact, irsogladine, irtemazole, isalsteine, isamoxole, isbogrel, isepamicin, isobengazole, isobutamben, isocarboxazid, isoconazole, isoetharine, isofloxythepin, isoflupredone acetate, isoflurane, isofluorophate, isohomohalicondrin B, isoleucine, isomazole hydrochloride, isomylamine hydrochloride, isoniazid, isopropamide iodide, isopropyl alcohol, isopropyl unoprostone, isoproterenol hydrochloride, isosorbide, isosorbide mononitrate, isotiquimide, isotretinoin, isoxepac, isoxicam, isoxsuprine hydrochloride, isradipine, itameline, itasetron, itazigrel, itopride, itraconazole, ivermectin, jasplakinolide, josamycin, kahalalide F, kalafungin, kanamycin sulfate, ketamine hydrochloride, ketanserin, ketazocine, ketazolam, kethoxal, ketipramine fumarate, ketoconazole, ketoprofen, ketorfanol, ketorolac, ketotifen fumarate, kitasamycin, labetalol hydrochloride, lacidipine, lacidipine, lactitol, lactivicin, laennec, lafutidine, lamellarin-n triacetate, lamifiban, lamivudine, lamotrigine, lanoconazole, LANOXIN® (digoxin, available from GlaxoSmithKline), lanperisone, lanreotide, lansoprazole (commercially available as PREVAID® from Takeda Pharmaceuticals, Inc.), latanoprost, lateritin, laurocapram, lauryl isoquinolinium bromide, lavoltidine succinate, lazabemide, lecimibide, leinamycin, lemildipine, leminoprazole, lenercept, leniquinsin, lenograstim, lenperone, lentinan sulfate, leptin, leptolstatin, lercanidipine, lergotrile, lerisetron, letimide hydrochloride, letrazuril, letrozole, leucine, leucomyzin, leuprolide acetate, leuprolide, leuprorelin, levamfetamine succinate, levamisole, levdobutamine lactobionate, levcromakalim, levetiracetam, levobetaxolol, levobunolol, levobupivacaine, levocabastine, levocarnitine, levodopa, levodropropizine, levofloxacin (commercially available as LEVAQUIN® from Jessen Pharmaceuticals, Inc.), levofuraltadone, levoleucovorin calcium, levomethadyl acetate, levomethadyl acetate hydrochloride, levomoprolol, levonantradol hydrochloride, levonordefrin, levonorgestrel, levopropoxyphene napsylate, levopropylcillin potassium, levormeloxifene, levorphanol tartrate, levosimendan, levosulpiride, levothyroxine sodium, levoxadrol hydrochloride, lexipafant, lexithromycin, liarozole, libenzapril, lidamidine hydrochloride, lidocaine, lidofenin, lidoflazine, lifarizine, lifibrate, lifibrol, linarotene, lincomycin, linear polyamine analogue, linogliride, linopirdine, linotroban, linsidomine, lintitript, lintopride, liothyronine I 125, liothyronine sodium, liotrix, lirexapride, lisinopril, lissoclinamide 7, lixazinone sulfate, lobaplatin, lobenzarit sodium, lobucavir, lodelaben, lodoxamide, lofemizole hydrochloride, lofentanil oxalate, lofepramine hydrochloride, lofexidine hydrochloride, lombricine, lomefloxacin, lomerizine, lometraline hydrochloride, lometrexol, lomitapide, lomofungin, lornoxicam, lomustine, lonapalene, lonazolac, lonidamine, loperamide hydrochloride, loracarbef, lorajmine hydrochloride, loratadine, lorazepam, lorbamate, lorcainide hydrochloride, loreclezole, lorglumide, lormetazepam, lornoxicam, lornoxicam, lortalamine, lorzafone, losartan (commercially available as COZAAR® from Merck), losigamone, losoxantrone, losulazine hydrochloride, loteprednol, lovastatin, loviride, loxapine, loxoribine, lubeluzole, lucanthone hydrochloride, lufironil, lurosetron mesylate, lurtotecan, luteinizing hormone, lutetium, lutrelin acetate, luzindole, lyapolate sodium, lycetamine, lydicamycin, lydimycin, lynestrenol, lypressin, lysine, lysofylline, lysostaphin, lytic peptides, maduramicin, mafenide, magainin 2 amide, magnesium salicylate, magnesium sulfate, magnolol, maitansine, malethamer, mallotochromene, mallotojaponin, malotilate, mangafodipir, manidipine, maniwamycin A, mannitol, mannostatin A, manumycin E, manumycin F, MAPK/ERK kinase (MEK) inhibitors, mapinastine, maprotiline, marimastat, masoprocol, maspin, massetolide, matrilysin inhibitors, maytansine, mazapertine succiniate, mazindol, mebendazole, mebeverine hydrochloride, mebrofenin, mebutamate, mecamylamine hydrochloride, mechlorethamine hydrochloride, meclocycline, meclofenamate sodium, mecloqualone, meclorisone dibutyrate, medazepam hydrochloride, medorinone, medrogestone, medroxalol, medroxyprogesterone (commercially available as DEPO-PROVERA® from Pfizer, Inc.), medrysone, meelizine hydrochloride, mefenamic acid, mefenidil, mefenorex hydrochloride, mefexamide, mefloquine hydrochloride, mefruside, megalomicin potassium phosphate, megestrol acetate, meglumine, meglutol, melengestrol acetate, melitracen hydrochloride, melphalan, memotine hydrochloride, menabitan hydrochloride, menoctone, menogaril, menotropins, meobentine sulfate, mepartricin, mepenzolate bromide, meperidine hydrochloride, mephentermine sulfate, mephenyloin, mephobarbital, mepivacaine hydrochloride, meprobamate, meptazinol hydrochloride, mequidox, meralein sodium, merbarone, mercaptopurine, mercufenol chloride, mercury, meropenem, mesalamine, meseclazone, mesoridazine, mesterolone, mestranol, mesuprine hydrochloride, metalol hydrochloride, metaproterenol polistirex, metaraminol bitartrate, metaxalone, meteneprost, meterelin, metformin, methacholine chloride, methacycline, methadone hydrochloride, methadyl acetate, methalthiazide, methamphetamine hydrochloride, methaqualone, methazolamide, methdilazine, methenamine, methenolone acetate, methetoin, methicillin sodium, methimazole, methioninase, methionine, methisazone, methixene hydrochloride, methocarbamol, methohexital sodium, methopholine, methotrexate, methotrimeprazine, methoxatone, methoxyflurane, methsuximide, methyclothiazide, methyl 10 palmoxirate, methylatropine nitrate, methylbenzethonium chloride, methyldopa, methyldopate hydrochloride, methylene blue, methylergonovine maleate, methylhistamine, R-alpha, methylinosine monophosphate, methylphenidate hydrochloride, methylprednisolone, methyltestosterone, methynodiol diacelate, methysergide, methysergide maleate, metiamide, metiapine, metioprim, metipamide, metipranolol, metizoline hydrochloride, metkephamid acetate, metoclopramide, metocurine iodide, metogest, metolazone, metopimazine, metoprine, metoprolol, metoquizine, metrifonate, metrizamide, metrizoate sodium, metronidazole, meturedepa, metyrapone, metyrosine, mexiletine hydrochloride, mexrenoate potassium, mezlocillin, mfonelic acid, mianserin hydrochloride, mibefradil, mibefradil dihydrochloride, mibolerone, michellamine B, miconazole, microcolin A, midaflur, midazolam hydrochloride, midodrine, mifepristone, mifobate, miglitol, milacemide, milameline, mildronate, milenperone, milipertine, milnacipran, milrinone, miltefosine, mimbane hydrochloride, minaprine, minaxolone, minocromil, minocycline, minoxidil, mioflazine hydrochloride, miokamycin, mipragoside, mirfentanil, mirimostim, mirincamycin hydrochloride, mirisetron maleate, mirtazapine, mismatched double stranded RNA, misonidazole, misoprostol, mitindomide, mitocarcin, mitocromin, mitogillin, mitoguazone, mitolactol, mitomalcin, mitomycin, mitonafide, mitosper, mitotane, mitoxantrone, mivacurium chloride, mivazerol, mixanpril, mixidine, mizolastine, mizoribine, moclobemide, modafinil, modaline sulfate, modecamide, moexipril, mof arotene, mofegiline hydrochloride, mofezolac, molgramostim, molinazone, molindone hydrochloride, molsidomine, mometasone, monatepil maleate, monensin, monoctanoin, montelukast sodium (commercially available as SINGULAIR® available from Merck), montirelin, mopidamol, moracizine, morantel tartrate, moricizine, morniflumate, morphine, morphine sulfate, morrhuate sodium, mosapramine, mosapride, motilide, motretinide, moxalactam disodium, moxazocine, moxiraprine, moxnidazole, moxonidine, mumps skin test antigen, mustard anticancer agent, muzolimine, mycaperoxide B, mycophenolic acid, myriaporone, nabazenil, nabilone, nabitan hydrochloride, naboctate hydrochloride, nabumetone, n-acetyldinaline, nadide, nadifloxacin, nadolol, nadroparin calcium, nafadotride, nafamostat, nafarelin, nafcillin sodium, nafenopin, nafimidone hydrochloride, naflocort, nafomine malate, nafoxidine hydrochloride, nafronyl oxalate, naftifine hydrochloride, naftopidil, naglivan, nagrestip, nalbuphine hydrochloride, nalidixate sodium, nalidixic acid, nalmefene, nalmexone hydrochloride, naloxone/pentazocine, naltrexone, namoxyrate, nandrolone phenpropionate, nantradol hydrochloride, napactadine hydrochloride, napadisilate, napamezole hydrochloride, napaviin, naphazoline hydrochloride, naphterpin, naproxen, naproxol, napsagatran, naranol hydrochloride, narasin, naratriptan, nartograstim, nasaruplase, natamycin, nateplase, naxagolide hydrochloride, nebivolol, nebramycin, nedaplatin, nedocromil, nefazodone hydrochloride, neflumozide hydrochloride, nefopam hydrochloride, nelezaprine maleate, nemazoline hydrochloride, nemorubicin, neomycin palmitate, neostigmine bromide, neridronic acid, netilmicin sulfate, neutral endopeptidase, neutramycin, nevirapine, nexeridine hydrochloride, niacin, nibroxane, nicardipine hydrochloride, nicergoline, niclosamide, nicorandil, nicotinyl alcohol, nicotine (commercially available as NICOTROL® NS from Pfizer, Inc.), nifedipine, nifirmerone, nifluridide, nifuradene, nifuraldezone, nifuratel, nifuratrone, nifurdazil, nifurimide, nifurpirinol, nifurquinazol, nifurthiazole, nilutamide, nilvadipine, nimazone, nimodipine, niperotidine, niravoline, niridazole, nisamycin, nisbuterol mesylate, nisin, nisobamate, nisoldipine, nisoxetine, nisterime acetate, nitarsone, nitazoxamide, nitecapone, nitrafudam hydrochloride, nitralamine hydrochloride, nitramisole hydrochloride, nitrazepam, nitrendipine, nitrocycline, nitrodan, nitrofurantoin, nitrofurazone, nitroglycerin, nitromersol, nitromide, nitromifene citrate, nitrous oxide, nitroxide antioxidant, nitrullyn, nivazol, nivimedone sodium, nizatidine, noberastine, nocodazole, nogalamycin, nolinium bromide, nomifensine maleate, noracymethadol hydrochloride, norbolethone, norepinephrine bitartrate, norethindrone, norethynodrel, norfloxacin, norflurane, norgestimate, norgestomet, norgestrel, nortriptyline hydrochloride, noscapine, novobiocin sodium, N-substituted benzaimides, nufenoxole, nylestriol, nystatin, O6-benzylguanine, obidoxime chloride, ocaperidone, ocfentanil hydrochloride, ocinaplon, octanoic acid, octazamide, octenidine hydrochloride, octodrine, octreotide, octriptyline phosphate, ofloxacin, oformine, okicenone, olanzapine (commercially available as ZYPREXA® from Eli Lilly and Company), oligonucleotides, olopatadine, olprinone, olsalazine, olsalazine sodium, olvanil, omeprazole, onapristone, ondansetron, ontazolast, oocyte maturation inhibitor, opipramol hydrochloride, oracin, orconazole nitrate, orgotein, orlislat, ormaplatin, ormetoprim, ornidazole, orpanoxin, orphenadrine citrate, osaterone, otenzepad, oxacillin sodium, oxagrelate, oxaliplatin, oxamarin hydrochloride, oxamisole, oxamniquine, oxandrolone, oxantel pamoate, oxaprotiline hydrochloride, oxaprozin, oxarbazole, oxatomide, oxaunomycin, oxazepam, oxcarbazepine, oxendolone, oxethazaine, oxetorone fumarate, oxfendazole, oxfenicine, oxibendazole, oxiconazole, oxidopamine, oxidronic acid, oxifungin hydrochloride, oxilorphan, oximonam, oximonam sodium, oxiperomide, oxiracetam, oxiramide, oxisuran, oxmetidine hydrochloride, oxodipine, oxogestone phenpropionate, oxolinic acid, oxprenolol hydrochloride, oxtriphylline, oxybutynin chloride, oxychlorosene, oxycodone, oxymetazoline hydrochloride, oxymetholone, oxymorphone hydrochloride, oxypertine, oxyphenbutazone, oxypurinol, oxytetracycline, oxytocin, ozagrel, ozolinone, paclitaxel, palauamine, paldimycin, palinavir, paliperidone (commercially available as INVEGA® from Janssen Pharmaceuticals, Inc.), paliperidone palmitate (commercially available as INVEGA® SUSTENNA® from Janssen Pharmaceuticals, Inc.), palmitoylrhizoxin, palmoxirate sodium, pamaqueside, pamatolol sulfate, pamicogrel, pamidronate disodium, pamidronic acid, panadiplon, panamesine, panaxytriol, pancopride, pancuronium bromide, panipenem, pannorin, panomifene, pantethine, pantoprazole, papaverine hydrochloride, parabactin, parachlorophenol, paraldehyde, paramethasone acetate, paranyline hydrochloride, parapenzolate bromide, pararosaniline pamoate, parbendazole, parconazole hydrochloride, paregoric, pareptide sulfate, pargyline hydrochloride, parnaparin sodium, paromomycin sulfate, paroxetine (commercially available as PAXIL® from GlaxoSmithKlein), parthenolide, partricin, paulomycin, pazelliptine, pazinaclone, pazoxide, pazufloxacin, pefloxacin, pegaspargase, pegorgotein, pelanserin hydrochloride, peldesine, peliomycin, pelretin, pelrinone hydrochloride, pemedolac, pemerid nitrate, pemetrexed, pemirolast, pemoline, penamecillin, penbutolol sulfate, penciclovir, penfluridol, penicillin G benzathine, penicillin G potassium, penicillin G procaine, penicillin G Sodium, penicillin V, penicillin V benzathine, penicillin V hydrabamine, penicillin V potassium, pentabamate, pentaerythritol tetranitrate, pentafuside, pentamidine, pentamorphone, bentamustine, pentapiperium methylsulfate, pentazocine, pentetic acid, pentiapine maleate, pentigetide, pentisomicin, pentizidone sodium, pentobarbital, pentomone, pentopril, pentosan, pentostatin, pentoxifylline, pentrinitrol, pentrozole, peplomycin sulfate, pepstatin, perflubron, perfof amide, perfosfamide, pergolide, perhexyline maleate, perillyl alcohol, perindopril, perindoprilat, perlapine, permethrin, perospirone, perphenazine, phenacemide, phenaridine, phenazinomycin, phenazopyridine hydrochloride, phenbutazone sodium glycerate, phencarbamide, phencyclidine hydrochloride, phendimetrazine tartrate, phenelzine sulfate, phenmetrazine hydrochloride, phenobarbital, phenoxybenzamine hydrochloride, phenprocoumon, phenserine, phensuccinal, phensuximide, phentermine, phentermine hydrochloride, phentolamine mesilate, phentoxifylline, phenyl aminosalicylate, phenylacetate, phenylalanine, phenylalanyl ketoconazole, phenylbutazone, phenylephrine hydrochloride, phenylpropanolamine hydrochloride, phenylpropanolamine polistirex, phenyramidol hydrochloride, phenyloin, phosphatase inhibitors, physostigmine, picenadol, picibanil, picotrin diolamine, picroliv, picumeterol, pidotimod, pifamine, pilocarpine, pilsicamide, pimagedine, pimetine hydrochloride, pimilprost, pimobendan, pimozide, pinacidil, pinadoline, pindolol, pinnenol, pinocebrin, pinoxepin hydrochloride, pioglitazone (commercially available as ACTOS® from Takeda Pharmaceuticals), pipamperone, pipazethate, pipecuronium bromide, piperacetazine, piperacillin sodium, piperamide maleate, piperazine, pipobroman, piposulfan, pipotiazine palmitate, pipoxolan hydrochloride, piprozolin, piquindone hydrochloride, piquizil hydrochloride, piracetam, pirandamine hydrochloride, pirarubicin, pirazmonam sodium, pirazolac, pirbenicillin sodium, pirbuterol acetate, pirenperone, pirenzepine hydrochloride, piretanide, pirfenidone, piridicillin sodium, piridronate sodium, piriprost, piritrexim, pirlimycin hydrochloride, pirlindole, pirmagrel, pirmenol hydrochloride, pirnabine, piroctone, pirodavir, pirodomast, pirogliride tartrate, pirolate, pirolazamide, piroxantrone hydrochloride, piroxicam, piroximone, pirprofen, pirquinozol, pirsidomine, prenylamine, pitavastatin (commercially available as LIVALOA® from Eli Lilly and Company), pituitary, posterior, pivampicillin hydrochloride, pivopril, pizotyline, placetin A, platinum compounds, platinum-triamine complex, plicamycin, plomestane, pobilukast edamine, podofilox, poisonoak extract, poldine methylsulfate, poliglusam, polignate sodium, polymyxin B sulfate, polythiazide, ponalrestat, porfimer sodium, porfiromycin, potassium chloride, potassium iodide, potassium permanganate, povidone-iodine, practolol, pralidoxime chloride, pramiracetam hydrochloride, pramoxine hydrochloride, pranolium chloride, prasugrel (commercially available as EFFIENT® from Eli Lilly and Company), pravadoline maleate, pravastatin, prazepam, prazosin, prazosin hydrochloride, prednazate, prednicarbate, prednimustine, prednisolone, prednisone, prednival, pregabalin (commercially available as LYRICA® from Pfizer, Inc.), pregnenolone succiniate, prenalterol hydrochloride, pridefine hydrochloride, prifelone, prilocalne hydrochloride, prilosec, primaquine phosphate, primidolol, primidone, prinivil, prinomide tromethamine, prinoxodan, prizidilol hydrochloride, proadifen hydrochloride, probenecid, probicromil calcium, probucol, procainamide hydrochloride, procaine hydrochloride, procarbazine hydrochloride, procaterol hydrochloride, prochlorperazine, procinonide, proclonol, procyclidine hydrochloride, prodilidine hydrochloride, prodolic acid, prof adol hydrochloride, progabide, progesterone, proglumide, proinsulin human, proline, prolintane hydrochloride, promazine hydrochloride, promethazine hydrochloride, propafenone hydrochloride, propagermanium, propanidid, propantheline bromide, proparacaine hydrochloride, propatyl nitrate, propentofylline, propenzolate hydrochloride, propikacin, propiomazine, propionic acid, propionylcarnitine, propiram, propiram+paracetamol, propiverine, propofol, propoxycaine hydrochloride, propoxyphene hydrochloride, propranolol hydrochloride, propulsid, propyl bis-acridone, propylhexedrine, propyliodone, propylthiouracil, proquazone, prorenoate potassium, proroxan hydrochloride, proscillaridin, prostalene, prostratin, protamine sulfate, protegrin, protirelin, protosufloxacin, protriptyline hydrochloride, proxazole, proxazole citrate, proxicromil, proxorphan tartrate, prulifloxacin, pseudoephedrine hydrochloride, desloratadine/pseudoephedrine sulfate (commercially available as CLARINEX-D® from Merck), puromycin, purpurins, pyrabrom, pyrantel, pamoate, pyrazinamide, pyrazofurin, pyrazoloacridine, pyridostigmine bromide, pyrilamine maleate, pyrimethamine, pyrinoline, pyrithione sodium, pyrithione zinc, pyrovalerone hydrochloride, pyroxamine maleate, pyrrocaine, pyrroliphene hydrochloride, pyrroinitrin, pyrvinium pamoate, quadazocine mesylate, quazepam, quazinone, quazodine, quazolast, quetiapine (commercially available as SEROQUEL® available from AstraZenica), quiflapon, quinagolide, quinaldine blue, quinapril, quinaprilat, quinazosin hydrochloride, quinbolone, quinctolate, quindecamine acetate, quindonium bromide, quinelorane hydrochloride, quinestrol, quinfamide, quingestanol acetate, quingestrone, quinidine gluconate, quinielorane hydrochloride, quinine sulfate, quinpirole hydrochloride, quinterenol sulfate, quinuclium bromide, quinupristin, quipazine maleate, rabeprazole sodium, racephenicol, racepinephrine, raf antagonists, rafoxanide, ralitoline, raloxifene, raltitrexed, ramatroban, ramipril, ramoplanin, ramosetron, ranelic acid, ranimycin, ranitidine, ranolazine, rauwolfia serpentina, recainam, recainam hydrochloride, reclazepam, regavirumab, regramostim, relaxin, relomycin, remacemide hydrochloride, remifentanil hydrochloride, remiprostol, remoxipride, repirinast, repromicin, reproterol hydrochloride, reserpine, resinferatoxin, resorcinol, retelliptine demethylated, reticulon, reviparin sodium, revizinone, rhenium re 186 etidronate, rhizoxin, ribaminol, ribavirin, riboprine, ribozymes, ricasetron, ridogrel, rifabutin, rifametane, rifamexil, rifamide, rifampin, rifapentine, rifaximin, retinamide, rilopirox, riluzole, rimantadine, rimcazole hydrochloride, rimexolone, rimiterol hydrobromide, rimoprogin, riodipine, rioprostil, ripazepam, ripisartan, risedronate sodium, risedronic acid, risocaine, risotilide hydrochloride, rispenzepine, risperdal, risperidone, ritanserin, ritipenem, ritodrine, ritolukast, ritonavir, rizatriptan benzoate, rocastine hydrochloride, rocuronium bromide, rodocaine, roflurane, rogletimide, rohitukine, rokitamycin, roletamicide, rolgamidine, rolicyprine, rolipram, rolitetracycline, rolodine, romazarit, romurtide, ronidazole, ropinirole (commercially available as REQUIP® from GlaxoSmithKline), ropitoin hydrochloride, ropivacaine, ropizine, roquinimex, rosaramicin, rosoxacin, rotoxamine, rosuvastatin (commercially available as CRESTOR® available from AstraZeneca), roxaitidine, roxarsone, roxindole, roxithromycin, rubiginone B1, ruboxyl, rufloxacin, rupatidine, rutamycin, ruzadolane, sabeluzole, safingol, safironil, saintopin, salbutamol, salcolex, salethamide maleate, salicyl alcohol, salicylamide, salicylate meglumine, salicylic acid, salmeterol, salnacediin, salsalate, sameridine, sampatrilat, sancycline, sanfetrinem, sanguinarium chloride, saperconazole, saprisartan, sapropterin, saquinavir, sarafloxacin hydrochloride, saralasin acetate, SarCNU, sarcophytol A, sargramostim, sarmoxicillin, sarpicillin, sarpogrelate, saruplase, saterinone, satigrel, satumomab pendetide, schick test control, scopafungin, scopolamine hydrobromide, scrazaipine hydrochloride, sdi 1 mimetics, secalciferol, secobarbital, seelzone, seglitide acetate, selegiline, selegiline hydrochloride, selenium sulfide, selenomethionine se 75, selfotel, sematilide, semduramicin, semotiadil, semustine, sense oligonucleotides, sepazonium chloride, seperidol hydrochloride, seprilose, seproxetine hydrochloride, seractide acetate, sergolexole maleate, serine, sermetacin, sermorelin acetate, sertaconazole, sertindole, sertraline, setiptiline, setoperone, sevirumab, sevoflurane, sezolamide, sibopirdine, sibutramine hydrochloride, signal transduction inhibitors, silandrone, sildenafil (commercially available as VIAGRA® from Pfizer Inc.), silipide, silteplase, silver nitrate, simendan, simtrazene, simvastatin (commercially available as ZOCOR® from Merck), sincalide, sinefungin, sinitrodil, sinnabidol, sipatrigine, sirolimus, sisomicin, sitogluside, sizofuran, sobuzoxane, sodium amylosulfate, sodium iodide I 123, sodium nitroprusside, sodium oxybate, sodium phenylacetate, sodium salicylate, solverol, solypertine tartrate, somalapor, somantadine hydrochloride, somatomedin B, somatomedin C, somatrem, somatropin, somenopor, somidobove, sonermin, sorbinil, sorivudine, sotalol, soterenol hydrochloride, sparfloxacin, sparfosate sodium, sparfosic acid, sparsomycin, sparteine sulfate, spectinomycin hydrochloride, spicamycin D, spiperone, spiradoline mesylate, spiramycin, spirapril hydrochloride, spiraprilat, spirogermanium hydrochloride, spiromustine, spironolactone, spiroplatin, spiroxasone, splenopentin, spongistatin 1, sprodiamide, squalamine, stallimycin hydrochloride, stannous pyrophosphate, stannous sulfur colloid, stanozolol, statolon, staurosporine, stavudine, steffimycin, stenbolone acetate, stepronin, stilbazium iodide, stilonium iodide, stipiamide, stiripentol, stobadine, streptomycin sulfate, streptonicozid, streptonigrin, streptozocin, stromelysin inhibitors, strontium chloride Sr 89, succibun, succimer, succinylcholine chloride, sucralfate, sucrosof ate potassium, sudoxicam, sufentanil, sufotidine, sulazepam, sulbactam pivoxil, sulconazole nitrate, sulfabenz, sulfabenzamide, sulfacetamide, sulfacytine, sulfadiazine, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfazamet, sulfinalol hydrochloride, sulfinosine, sulfinpyrazone, sulfisoxazole, sulfomyxin, sulfonterol hydrochloride, sulfoxamine, sulinldac, sulmarin, sulnidazole, suloctidil, sulofenur, sulopenem, suloxifen oxalate, sulpiride, sulprostone, sultamicillin, sulthiame, sultopride, sulukast, sumarotene, sumatriptan, suncillin sodium, suproclone, suprofen, suradista, suramin, surfomer, suricamide maleate, suritozole, suronacrine maleate, suxemerid sulfate, swainsonine, symakalim, symclosene, symetine hydrochloride, synthetic glycosaminoglycans, tadalafil (commercially available as CIALIS® and ACIRCA® from from Eli Lilly and Company), taciamine hydrochloride, tacrine hydrochloride, tacrolimus, talampicillin hydrochloride, taleranol, talisomycin, tallimustine, talmetacin, talniflumate, talopram hydrochloride, talosalate, tametraline hydrochloride, tamoxifen (commercially available as NOLVADEX® from AstraZeneca), tampramine fumarate, tamsulosin hydrochloride, tandamine hydrochloride, tandospirone, tapgen, taprostene, tasosartan, tauromustine, taxane, taxoid, tazadolene succinate, tazanolast, tazarotene, tazifylline hydrochloride, tazobactam, tazofelone, tazolol hydrochloride, tebufelone, tebuquine, technetium Tc 99 m bicisate, teclozan, tecogalan sodium, teecleukin, teflurane, tegafur, tegretol, teicoplanin, telenzepine, tellurapyrylium, telmesteine, telmisartan, telomerase inhibitors, teloxantrone hydrochloride, teludipine hydrochloride, temafloxacin hydrochloride, tematropium methyl sulfate, temazepam, temelastine, temocapril, temocillin, temoporfin, temozolomide, tenofovir, tenidap, teniposide, tenosal, tenoxicam, tepirindole, tepoxalin, teprotide, terazosin, terbinafine, terbutaline sulfate (commercially available as BRICANYL® from AstraZeneca), terconazole, terfenadine, terflavoxate, terguride, teriparatide acetate, terlakiren, terlipressin, terodiline, teroxalene hydrochloride, teroxirone, tertatolol, tesicam, tesimide, testolactone, testosterone, tetracaine, tetrachlorodecaoxide, tetracycline, tetrahydrozoline hydrochloride, tetramisole hydrochloride, tetrazolast meglumine, tetrazomine, tetrofosmin, tetroquinone, tetroxoprim, tetrydamine, thaliblastine, thalidomide, theofibrate, theophylline, thiabendazole, thiamiprine, thiamphenicol, thiamylal, thiazesim hydrochloride, thiazinamium chloride, thiazolidinedione, thiethylperazine, thimerfonate sodium, thimerosal, thiocoraline, thiofedrine, thioguanine, thiomarinol, thiopental sodium, thioperamide, thioridazine, thiotepa, thiothixene, thiphenamil hydrochloride, thiphencillin potassium, thiram, thozalinone, threonine, thrombin, thrombopoietin, thrombopoietin mimetic, thymalfasin, thymopoietin receptor agonist, thymotrinan, thyromedan hydrochloride, thyroxine 1 125, thyroxine 1 131, tiacrilast, tiacrilast sodium, tiagabine, tiamenidine, tianeptine, tiapafant, tiapamil hydrochloride, tiaramide hydrochloride, tiazofurin, tibenelast sodium, tibolone, tibric acid, ticabesone propionate, ticarbodine, ticarcillin cresyl sodium, ticlatone, ticlopidine, ticrynafen, tienoxolol, tifurac sodium, tigemonam dicholine, tigestol, tiletamine hydrochloride, tilidine hydrochloride, tilisolol, tilnoprofen arbamel, tilorone hydrochloride, tiludronate disodium, tiludronic acid, timefurone, timobesone acetate, timolol, tin ethyl etiopurpurin, tinabinol, tinidazole, tinzaparin sodium, tioconazole, tiodazosin, tiodonium chloride, tioperidone hydrochloride, tiopinac, tiospirone hydrochloride, tiotidine, tiotropium bromide, tioxidazole, tipentosin hydrochloride, tipredane, tiprenolol hydrochloride, tiprinast meglumine, tipropidil hydrochloride, tiqueside, tiquinamide hydrochloride, tirandalydigin, tirapazamine, tirilazad, tirofiban, tiropramide, titanocene dichloride, tixanox, tixocortol pivalate, tizanidine hydrochloride, tobramycin, tocainide, tocamphyl, tofenacin hydrochloride, tolamolol, tolazamide, tolazoline hydrochloride, tolbutamide, tolcapone, tolciclate, tolfamide, tolgabide, lamotrigine, tolimidone, tolindate, tolmetin, tolnaftate, tolpovidone 1 131, tolpyrramide, tolrestat, tomelukast, tomoxetine hydrochloride, tonazocine mesylate, topiramate, topotecan, topotecan hydrochloride, topsentin, topterone, toquizine, torasemide, toremifene, torsemide, tosifen, tosufloxacin, totipotent stem cell factor, tracazolate, trafermin, tralonide, tramadol hydrochloride, tramazoline hydrochloride, trandolapril, tranexamic acid, tranilast, transcamide, translation inhibitors, trastuzumab (commercially available as HERCEPTIN® from Genentech), traxanox, trazodone hydrochloride, trazodone-hcl, trebenzomine hydrochloride, trefentanil hydrochloride, treloxinate, trepipam maleate, trestolone acetate, tretinoin, triacetin, triacetyluridine, triafungin, triamcinolone, triampyzine sulfate, triamterene, triazolam, tribenoside, tricaprilin, tricetamide, trichlormethiazide, trichohyalin, triciribine, tricitrates, triclofenol piperazine, triclofos sodium, triclonide, trientine, trifenagrel, triflavin, triflocin, triflubazam, triflumidate, trifluoperazine hydrochloride, trifluperidol, triflupromazine, triflupromazine hydrochloride, trifluridine, trihexyphenidyl hydrochloride, trilostane, trimazosin hydrochloride, trimegestone, trimeprazine tartrate, trimethadione, trimethaphan camsylate, trimethobenzamide hydrochloride, trimethoprim, trimetozine, trimetrexate, trimipramine, trimoprostil, trimoxamine hydrochloride, triolein 1 125, triolein 1 131, trioxifene mesylate, tripamide, tripelennamine hydrochloride, triprolidine hydrochloride, triptorelin, trisulfapyrimidines, troclosene potassium, troglitazone, trolamine, troleandomycin, trombodipine, trometamol, tropanserin hydrochloride, tropicamide, tropine ester, tropisetron, trospectomycin, trovafloxacin, trovirdine, tryptophan, tuberculin, tubocurarine chloride, tubulozole hydrochloride, tucarcsol, tulobuterol, turosteride, tybamate, tylogenin, tyropanoate sodium, tyrosine, tyrothricin, tyrphostins, ubenimex, uldazepam, undecylenic acid, uracil mustard, urapidil, urea, uredepa, uridine triphosphate, urofollitropin, urokinase, ursodiol, valaciclovir, valine, valnoctamide, valproate sodium, valproic acid, valsartan (commercially available as DIOVAN® from Novartis Pharmaceuticals), vamicamide, vanadeine, vancomycin, vaminolol, vapiprost hydrochloride, vapreotide, vardenafil (commercially available as LEVITRA® from GlaxoSmithKline), variolin B, vasopressin, vecuronium bromide, velaresol, velnacrine maleate, venlafaxine, veradoline hydrochloride, veramine, verapamil hydrochloride, verdins, verilopam hydrochloride, verlukast, verofylline, veroxan, verteporfin, vesnarinone, vexibinol, vidarabine, vigabatrin, viloxazine hydrochloride, vinblastine sulfate, vinburnine citrate, vincofos, vinconate, vincristine sulfate, vindesine, vindesine sulfate, vinepidine sulfate, vinglycinate sulfate, vinleurosine sulfate, vinorelbine, vinpocetine, vintoperol, vinxaltine, vinzolidine sulfate, viprostol, virginiamycin, viridofulvin, viroxime, vitaxin, volazocine, voriconazole, vorozole, voxergolide, warfarin sodium, xamoterol, xanomeline, xanoxate sodium, xanthinol niacinate, xemilofiban, xenalipin, xenbucin, xilobam, ximoprofen, xipamide, xorphanol mesylate, xylamidine tosylate, xylazine hydrochloride, xylometazoline hydrochloride, xylose, yangambin, zabicipril, zacopride, zafirlukast, zalcitabine, zaleplon, zalospirone, zaltidine hydrochloride, zaltoprofen, zanamivir, zankiren, zanoterone, zantac, zarirlukast, zatebradine, zatosetron, zatosetron maleate, zenarestat, zenazocine mesylate, zeniplatin, zeranol, zidometacin, zidovudine, zifrosilone, zilantel, zilascorb, zileuton, zimeldine hydrochloride, zinc undecylenate, zindotrine, zinoconazole hydrochloride, zinostatin, zinterol hydrochloride, zinviroxime, ziprasidone, zobolt, zofenopril calcium, zofenoprilat, zolamine hydrochloride, zolazepam hydrochloride, zoledronie acid, zolertine hydrochloride, zolmitriptan, zolpidem, zomepirac sodium, zometapine, zoniclezole hydrochloride, zonisamide, zopiclone, zopolrestat, zorbamyciin, zorubicin hydrochloride, zotepine, zucapsaicin, JTT-501 (PNU-182716) (reglitazar), AR-H039122, MCC-555 (netoglitazone), AR-H049020 (tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (ragaglitazar), L-686398, L-168049, L-805645, L-054852, demethyl asteriquinone B1 (L-783281), L-363586, KRP-297, P32/98, CRE-16336, EML-1625, pharmaceutically acceptable salts thereof (e.g., Zn, Fe, Mg, K, Na, F, Cl, Br, I, acetate, diacetate, nitrate, nitrite, sulfate, sulfite, phosphate, and phosphite salts), pharmaceutically acceptable forms thereof with acid associates (e.g. HCl), and any combination thereof.

Examples of antibiotics may include, but are not limited to, to β-lactam antibiotics (e.g., benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (amoxicillin+clavulanic acid), azlocillin, carbenicillin, ticarcillin, mezlocillin, piperacillin, cephalosporin, cephalexin, cephalothin, cefazolin, cefaclor, cefuroxime, cefamandole, cefotetan, cefoxitin, ceftriaxone, cefotaxime, cefpodoxime, cefixime, ceftazidime, cefepime, cefpirome, carbapenem, imipenem (with cilastatin), meropenem, ertapenem, faropenem, doripenem, aztreonam (commercially available as AZACTAM® from Bristol-Myers Squibb), tigemonam, nocardicin A, tabtoxinine-β-lactam, clavulanic acid, tazobactam, and sulbactam); aminoglycoside antibiotics (e.g., aminoglycoside, amikacin, apramycin, arbekacin, astromicin, bekanamycin, capreomycin, dibekacin, dihydrostreptomycin, elsamitrucin, G418, gentamicin, hygromycin B, isepamicin, kanamycin, kasugamycin, micronomicin, neomycin, netilmicin, paromomycin sulfate, ribostamycin, sisomicin, streptoduocin, streptomycin, tobramycin, verdamicin; sulfonamides such as sulfamethoxazole, sulfisomidine (also known as sulfaisodimidine), sulfacetamide, sulfadoxine, dichlorphenamide (DCP), and dorzolamide); quinolone antibiotics (e.g., cinobac, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, garenoxacin, and delafloxacin); oxazolidone antibiotics (e.g., linezolid, torezolid, eperezolid, posizolid, and radezolid), and any combination thereof.

Examples of antifungals suitable may include, but are not limited to, polyene antifungals (e.g., natamycin, rimocidin, filipin, nystatin, amphotericin B, candicin, and hamycin; imidazole antifungals such as miconazole (commercially available as MICATIN® from WellSpring Pharmaceutical Corporation), ketoconazole (commercially available as NIZORAL® from McNeil consumer Healthcare), clotrimazole (commercially available as LOTRAMIN® and LOTRAMIN AF® available from Merck and CANESTEN® available from Bayer), econazole, omoconazole, bifonazole, butoconazole, fenticonazole, isoconazole, oxiconazole, sertaconazole (commercially available as ERTACZO® from OrthoDematologics), sulconazole, and tioconazole; triazole antifungals such as fluconazole, itraconazole, isavuconazole, ravuconazole, posaconazole, voriconazole, terconazole, and albaconazole), thiazole antifungals (e.g., abafungin), allylamine antifungals (e.g., terbinafine (commercially available as LAMISIL® from Novartis Consumer Health, Inc.), naftifine (commercially available as NAFTIN® available from Merz Pharmaceuticals), and butenafine (commercially available as LOTRAMIN ULTRA® from Merck), echinocandin antifungals (e.g., anidulafungin, caspofungin, and micafungin), polygodial, benzoic acid, ciclopirox, tolnaftate (e.g., commercially available as TINACTIN® from MDS Consumer Care, Inc.), undecylenic acid, flucytosine, 5-fluorocytosine, griseofulvin, haloprogin, and any combination thereof.

Examples of active biologicals may include, but are not limited to, hormones (synthetic or natural and patient derived or otherwise), DNAs (synthetic or natural and patient derived or otherwise), RNAs (synthetic or natural and patient derived or otherwise), siRNAs (synthetic or natural and patient derived or otherwise), proteins and peptides (e.g., albumin, atrial natriuretic factor, renin, superoxide dismutase, α1-antitrypsin, lung surfactant proteins, bacitracin, bestatin, cydosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-α-endorphin, gonadotropin releasing hormone, leuprolide, α-MSH, and metkephamid), enzymes, nucleotides, oligionucleotides, antibodies, monoclonal antibodies, growth factors (e.g., epidermal growth factor (EGF), fibroblast growth factors, basic fibroblast growth factor (bFGF), nerve growth factor (NGF), bone derived growth factor (BDGF), transforming growth factors, transforming growth factor-β1 (TGF-β1), and human growth gormone (hGH)), viral surface antigens (e.g., adenoviruses, epstein-barr virus, hepatitis A virus, hepatitis B virus, herpes viruses, HIV-1, HIV-2, HTLV-III, influenza viruses, Japanese encephalitis virus, measles virus, papilloma viruses, paramyxoviruses, polio virus, rabies virus, rubella virus, vaccinia (smallpox) viruses, and yellow fever virus), bacterial surface antigens (e.g., bordetella pertussis, helicobacter pylorn, clostridium tetani, corynebacterium diphtheria, escherichia coli, haemophilus influenza, klebsiella species, legionella pneumophila, mycobacterium bovis, mycobacterium leprae, mycrobacterium tuberculosis, neisseria gonorrhoeae, neisseria meningitidis, proteus species, pseudomonas aeruginosa, salmonella species, shigella species, staphylococcus aureus, streptococcus pyogenes, vibrio cholera, and yersinia pestis), parasite surface antigens (e.g., plasmodium vivax—malaria, plasmodium falciparum—malaria, plasmodium ovale—malaria, plasmodium malariae—malaria, leishmania tropica—leishmaniasis, leishmania donovani, leishmaniasis, leishmania branziliensis—leishmaniasis, trypanosoma rhodescense—sleeping sickness, trypanosoma gambiense—sleeping sickness, trypanosoma cruzi—Chagas' disease, schistosoma mansoni—schistosomiasis, schistosomoma haematobium—schistomiasis, schistosoma japonicum—shichtomiasis, trichinella spiralis—trichinosis, stronglyloides duodenale—hookworm, ancyclostoma duodenale—hookworm, necator americanus—hookworm, wucheria bancrofti—filariasis, brugia malaya—filariasis, loa loa—filariasis, dipetalonema perstaris—filariasis, dracuncula medinensis—filariasis, and onchocerca volvulus—filariasis), immunogobulins (e.g., IgG, IgA, IgM, antirabies immunoglobulin, and antivaccinia immunoglobulin), and any combination thereof.

Examples of antitoxins may include, but are not limited to, botulinum antitoxin, diphtheria antitoxin, gas gangrene antitoxin, tetanus antitoxin, and any combination thereof.

Examples of antigents may include, but are not limited to, foot and mouth disease, hormones and growth factors (e.g., follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone), cytokines (e.g., interferons, interleukins, colony stimulating factors, and tumor necrosis factors: fibrinolytic enzymes, such as urokinase, kidney plasminogen activator), clotting factors (e.g., Protein C, Factor VIII, Factor IX, Factor VII and Antithrombin III), and any combination thereof.

Examples of nutritional supplements may include, but are not limited to, vitamins, minerals, herbs, botanicals, amino acids, steroids, and the like.

Examples of imaging agents may include, but are not limited to, iron oxide, gadolinium ions, iodine, perfluorocarbons, radioisotopes, and the like.

Examples of fluid stabilizers may include, but are not limited to, at least one component of citrate phosphate with dextrose buffer (e.g., stabilizing blood), blood clotting factors, emulsion stabilizers, antifoamers, agar, pectin, and the like, and any combination thereof.

Examples of food agents may include, but are not limited to, caffeine, flavors, aromas, vitamins, minerals, herbs, minerals, antioxidants, calcium propionate, sodium nitrate, sodium nitrite, sulfites, sulfur dioxide, sodium bisulfite, potassium hydrogen sulfite, disodium EDTA, salt, rosemary extract, sugar, low-calorie sweeteners, no-calorie sweeteners, vinegar, alcohol, hops, diatomaceous earth, and the like, and any combination thereof.

Examples of nutraceuticals may include, but are not limited to, dietary supplements, botanicals, functional foods and extracts thereof, medicinal foods and extracts thereof, vitamins, minerals, co-enzyme Q, carnitine, multi-mineral formulas, ginseng, gingko biloba, saw palmetto, other plant-based supplements, probiotics, omega-3, canola and other oils, plant stanols, natural sweeteners, mushroom extracts, chocolate, chocolate extracts, grape extracts, berry extracts, super food extracts, quillaja molina extracts, plant extracts, yucca schidigera extract, bran, alanine, beta-carotene, carotenoids, arginin, vitamin A, asparagine, vitamin B-complex, aspartate, vitamin C, leucine, isoleucine, valine, vitamin D, citrulline, vitamin E, cysteine, vitamin K, glutamine, minerals, micro-nutrients, glutamic acid, calcium, glycine, chromium, histidine, copper, lysine, iodine, methionine, iron, ornithine, magnesium, phenylalanine, potassium, proline, selenium, serine, zinc, taurine, threonine, alpha lipoic acid, tryptophan, green tea extracts, tyrosine, essential fatty acids (EFA), whey protein, flax seed oil, and any combination thereof.

Examples of olfactory agents may include, but are not limited to, spices, spice extracts, herb extracts, essential oils, smelling salts, volatile organic compounds, volatile small molecules, methyl formate, methyl acetate, methyl butyrate, ethyl acetate, ethyl butyrate, isoamyl acetate, pentyl butyrate, pentyl pentanoate, octyl acetate, myrcene, geraniol, nerol, citral, citronellal, citronellol, linalool, nerolidol, limonene, camphor, terpineol, alpha-ionone, thujone, benzaldehyde, eugenol, cinnamaldehyde, ethyl maltol, vanilla, anisole, anethole, estragole, thymol, furaneol, methanol, rosemary, lavender, citrus, freesia, apricot blossoms, greens, peach, jasmine, rosewood, pine, thyme, oakmoss, musk, vetiver, myrrh, blackcurrant, bergamot, grapefruit, acacia, passiflora, sandalwood, tonka bean, mandarin, neroli, violet leaves, gardenia, red fruits, ylang-ylang, acacia farnesiana, mimosa, tonka bean, woods, ambergris, daffodil, hyacinth, narcissus, black currant bud, iris, raspberry, lily of the valley, sandalwood, vetiver, cedarwood, neroli, bergamot, strawberry, carnation, oregano, honey, civet, heliotrope, caramel, coumarin, patchouli, dewberry, helonial, bergamot, hyacinth, coriander, pimento berry, labdanum, cassie, bergamot, aldehydes, orchid, amber, benzoin, orris, tuberose, palmarosa, cinnamon, nutmeg, moss, styrax, pineapple, bergamot, foxglove, tulip, wisteria, clematis, ambergris, gums, resins, civet, peach, plum, castoreum, civet, myrrh, geranium, rose violet, jonquil, spicy carnation, galbanum, hyacinth, petitgrain, iris, hyacinth, honeysuckle, pepper, raspberry, benzoin, mango, coconut, hesperides, castoreum, osmanthus, mousse de chene, nectarine, mint, anise, cinnamon, orris, apricot, plumeria, marigold, rose otto, narcissus, tolu balsam, frankincense, amber, orange blossom, bourbon vetiver, opopanax, white musk, papaya, sugar candy, jackfruit, honeydew, lotus blossom, muguet, mulberry, absinthe, ginger, juniper berries, spicebush, peony, violet, lemon, lime, hibiscus, white rum, basil, lavender, balsamics, fo-ti-tieng, osmanthus, karo karunde, white orchid, calla lilies, white rose, rhubrum lily, tagetes, ambergris, ivy, grass, seringa, spearmint, clary sage, cottonwood, grapes, brimbelle, lotus, cyclamen, orchid, glycine, tiare flower, ginger lily, green osmanthus, passion flower, blue rose, bay rum, cassie, African tagetes, Anatolian rose, Auvergne narcissus, British broom, British broom chocolate, Bulgarian rose, Chinese patchouli, Chinese gardenia, Calabrian mandarin, Comoros Island tuberose, Ceylonese cardamom, Caribbean passion fruit, Damascena rose, Georgia peach, white Madonna lily, Egyptian jasmine, Egyptian marigold, Ethiopian civet, Farnesian cassie, Florentine iris, French jasmine, French jonquil, French hyacinth, Guinea oranges, Guyana wacapua, Grasse petitgrain, Grasse rose, Grasse tuberose, Haitian vetiver, Hawaiian pineapple, Israeli basil, Indian sandalwood, Indian Ocean vanilla, Italian bergamot, Italian iris, Jamaican pepper, May rose, Madagascar ylang-ylang, Madagascar vanilla, Moroccan jasmine, Moroccan rose, Moroccan oakmoss, Moroccan orange blossom, Mysore sandalwood, Oriental rose, Russian leather, Russian coriander, Sicilian mandarin, South African marigold, South American tonka bean, Singapore patchouli, Spanish orange blossom, Sicilian lime, Reunion Island vetiver, Turkish rose, Thai benzoin, Tunisian orange blossom, Yugoslavian oakmoss, Virginian cedarwood, Utah yarrow, West Indian rosewood, and the like, and any combination thereof.

Examples of flavorants may include, but are not limited to, tobacco, menthol, cloves, cherry, chocolate, orange, mint, mango, vanilla, cinnamon, and the like. Such flavorants may, in some embodiments, be provided by menthol, anethole (licorice), anisole, limonene (citrus), eugenol (clove), a flavorant associated with an olfactory agent described herein, and the like, and any combination thereof.

Examples of plant agents may include, but are not limited to, herbicides, fungicides, insecticides, bactericides, nitrogen sources, phosphorous sources, potassium sources, calcium sources, magnesium sources, sulfur sources, boron sources, chlorine sources, copper sources, iron sources, manganese sources, molybdenum sources, zinc sources, saltpeter, growth promoters, hormones, and the like, and any combination thereof.

Examples of chemical-reaction agents may include, but are not limited to, positive catalysts, inhibitors, and the like, and any combination thereof.

As used herein, the term “insect repellent” refers to both insect repellents and insecticides. One skilled in the art with the benefit of this disclosure should understand that because controlled release vehicles described herein, in some embodiments, are designed to be administered to a patient, insect repellents should be chosen that are compatible with such a desired administration technique. Examples of insect repellents may include, but are not limited to, natural repellents (e.g., essential oils, citronella, sodium laurel sulfate, cedar, neem, clove, thyme, lavender, eucalyptus, peppermint, lemongrass, garlic, capsaicin, sabadillia, rotenone, nicotine, and pyrethrum), synthetic repellents (e.g., N,N-dimethyl-meta-toluamide (DEET), dichlorodiphenyltrichloroethane (DDT), organophosphate-based insecticides, pyrethroids, picaridin, boric acid, cyfluthrin, deltamethrin, fenthion, propoxur, sevin, dinotefuran, acephate, chlorophyrifos, diazinon, horticultural oil, malathion, and methoxyclor), insect controlling pheromones, and the like, and any combination thereof. Examples of insecticides may include, but are not limited to, acid copper chromate (ACC), acetamiprid, bifenazate, chlorantraniliprole, chlorfenapyr, clothianidin, dinotefuran, ethiprole, flubendiamide, flufenoxuron, imiprothrin, indoxacarb, metrafenone, nicarbazin, n-methylneodecanamide, phosphine, pirimicarb, pyridalyl, spinetoram, spinosad, spirodiclofen, spirotetramat, tebufenpyrad, thiacloprid, pyrethrin, allethrin, prallethrin, furamethrin, phenothrin, permethrin, imidacloprid, pyriproxyfen silafluofen, hinokitiol, isopropylmethyl phenol, 5-chloro-2-trifluoromethanesulfonamide methyl benzoate, taufluvalinate, flumethrin, trans-cyfluthrin, kadethrin, bioresmethrin, tetramethrin, empenthrin, cyphenothrin, bioallethrin, an oxadiazine derivative, a chloronicotinyl, a nitroguanidine, a pyrrol, a pyrazone, a diacylhydrazine, a triazole, a biological/fermentation product, a phenyl pyrazole, an organophosphate, a carbamate, a pyrethrin, d-trans allethrin, esbiol, esbiothrin, pynamin forte, n-octyl bicycloheptene dicarboximide, and the like, and any combination thereof. Further, an insect repellent may be utilized, in some embodiments, in conjunction with an insect repellent synergist, a chemical or biological compound that interferes with an insect's ability to mitigate the effects of an insect repellent. Examples of insect repellent synergists may include, but are not limited to, piperonyl butoxide, dietholate, sesamex, sulfoxide, butcarpolate, sesamolin, jiajizengxiaolin, octachlorodipropylether, piperonyl cyclonene, piprotal, propylisome, and any combination thereof. In some embodiments, an insect repellent, and preferably an insect repellant that comprises an insecticide, may be used in conjunction with compounds that attracts insects to a microsphere or article comprising microspheres described herein, including, but not limited to, any suitable aroma described herein.

Exemplary embodiment A disclosed herein includes: a method that includes forming a polysaccharide ester product from a polysaccharide synthesis, wherein the polysaccharide ester product comprises a polysaccharide ester and a solvent; diluting the polysaccharide ester product, thereby yielding a polysaccharide ester dope; and forming a plurality of polysaccharide ester microspheres from the polysaccharide ester dope. Optionally, at least one of the following elements may be included in the method: Element 1: the method further including filtering the polysaccharide ester product before diluting; Element 2: wherein the polysaccharide ester comprises at least one ester derivative of at least one selected from the group consisting of starch, cellulose, hemicellulose, algenates, chitosan, and any combination thereof; Element 3: Element 2 wherein the ester derivative comprises at least one organic ester substituent selected from the group consisting of an C₁-C₂₀ aliphatic ester, a functional C₁-C₂₀ aliphatic ester, an aromatic ester, a substituted aromatic ester, any derivative thereof, and any combination thereof; Element 4: Element 2 wherein the ester derivative comprises at least one inorganic ester substituent selected from the group consisting of hypochlorite, chlorite, chlorate, perchlorate, sulfite, sulfate, a sulfonate, fluorosulfate, nitrite, nitrate, phosphite, phosphate, phosphonates, borate, any derivative thereof, and any combination thereof; Element 5: wherein the polysaccharide ester comprises at least one ester derivative of starch and at least one ester derivative of cellulose; Element 6: Element 5 wherein the polysaccharide ester dope has a solids content of about 4% to about 50% by weight of the polysaccharide ester dope; and Element 7: wherein forming the plurality of polysaccharide ester microspheres involves spraying the polysaccharide ester dope into a coagulation bath comprising a non-solvent of the polysaccharide ester. Exemplary combinations of embodiments may include, but are not limited to, Element 1 in combination with Element 5; Element 3 in combination with Element 5; Element 3 in combination with Element 4; Element 4 in combination with Element 5; Elements 3 and 4 in combination with Element 5; Element 5 in combination with Element 6; Element 1 in combination with Element 6; Element 3 in combination with Element 6; Element 4 in combination with Element 6; Elements 3 and 4 in combination with Element 6; Element 1 in combination with Elements 5 and 6; Element 3 in combination with Elements 5 and 6; Element 4 in combination with Elements 5 and 6; Elements 3 and 4 in combination with Elements 5 and 6; and Element 7 in combination with any of the foregoing.

Exemplary embodiment B disclosed herein includes: a method that includes forming a polysaccharide ester product from a polysaccharide synthesis, wherein the polysaccharide ester product comprises a starch ester and a solvent; producing a polysaccharide ester dope from the polysaccharide ester product, wherein the polysaccharide ester dope has a solids content of about 16% to about 50% by weight of the polysaccharide ester dope; and forming a plurality of polysaccharide ester microspheres from the polysaccharide ester dope. Optionally, at least one of the following elements may be included in the method: Element 8: wherein the polysaccharide ester product further comprises a cellulose ester; Element 9: the method further comprising adding a cellulose ester to the polysaccharide ester dope; Element 10: wherein the polysaccharide ester further comprises at least one ester derivative of at least one selected from the group consisting of hemicellulose, algenates, chitosan, and any combination thereof; Element 11: wherein the ester derivative comprises at least one organic ester substituent selected from the group consisting of an C₁-C₂₀ aliphatic ester, a functional C₁-C₂₀ aliphatic ester, an aromatic ester, a substituted aromatic ester, any derivative thereof, and any combination thereof; Element 12: wherein the ester derivative comprises at least one inorganic ester substituent selected from the group consisting of hypochlorite, chlorite, chlorate, perchlorate, sulfite, sulfate, a sulfonate, fluorosulfate, nitrite, nitrate, phosphite, phosphate, phosphonates, borate, any derivative thereof, and any combination thereof; Element 13: wherein producing a polysaccharide ester dope involves diluting the polysaccharide ester product and optionally filtering the polysaccharide ester product before and/or after diluting; Element 14: wherein producing a polysaccharide ester dope involves finishing the polysaccharide ester product and suspending the finished polysaccharide ester product in a solvent; and Element 15: wherein forming the plurality of polysaccharide ester microspheres involves spraying the polysaccharide ester dope into a coagulation bath comprising a non-solvent of the polysaccharide ester. Exemplary combinations of embodiments may include, but are not limited to, one of Elements 8-10 in combination with Element 11; one of Elements 8-10 in combination with Element 12; Element 11 in combination with Element 12; Element 15 in combination with any of the foregoing; and one of Elements 13 and 14 in combination with any of the foregoing.

Exemplary embodiment C disclosed herein includes: a method that includes providing a finished polysaccharide ester product that comprises a starch ester; producing a polysaccharide ester dope that comprises the finished polysaccharide ester product and a solvent, wherein the polysaccharide ester dope has a solids content of about 16% to about 50% by weight of the polysaccharide ester dope; and forming a plurality of polysaccharide ester microspheres from the polysaccharide ester dope. Optionally, at least one of the following elements may be included in the method: Element 8: wherein the polysaccharide ester product further comprises a cellulose ester; Element 9: the method further comprising adding a cellulose ester to the polysaccharide ester dope; Element 10: wherein the polysaccharide ester further comprises at least one ester derivative of at least one selected from the group consisting of hemicellulose, algenates, chitosan, and any combination thereof; Element 11: wherein the ester derivative comprises at least one organic ester substituent selected from the group consisting of an C₁-C₂₀ aliphatic ester, a functional C₁-C₂₀ aliphatic ester, an aromatic ester, a substituted aromatic ester, any derivative thereof, and any combination thereof; Element 12: wherein the ester derivative comprises at least one inorganic ester substituent selected from the group consisting of hypochlorite, chlorite, chlorate, perchlorate, sulfite, sulfate, a sulfonate, fluorosulfate, nitrite, nitrate, phosphite, phosphate, phosphonates, borate, any derivative thereof, and any combination thereof; and Element 15: wherein forming the plurality of polysaccharide ester microspheres involves spraying the polysaccharide ester dope into a coagulation bath comprising a non-solvent of the polysaccharide ester. Exemplary combinations of embodiments may include, but are not limited to, one of Elements 8-10 in combination with Element 11; one of Elements 8-10 in combination with Element 12; Element 11 in combination with Element 12; and Element 15 in combination with any of the foregoing.

Exemplary embodiment D disclosed herein includes: a microsphere that includes a starch ester and having a hollow core and a wall. Optionally, at least one of the following elements may be included in the composition: Element 16: wherein the starch ester comprises at least one organic ester substituent selected from the group consisting of an C₁-C₂₀ aliphatic ester, a functional C₁-C₂₀ aliphatic ester, an aromatic ester, a substituted aromatic ester, any derivative thereof, and any combination thereof; Element 17: wherein the starch ester comprises at least one inorganic ester substituent selected from the group consisting of hypochlorite, chlorite, chlorate, perchlorate, sulfite, sulfate, a sulfonate, fluorosulfate, nitrite, nitrate, phosphite, phosphate, phosphonates, borate, any derivative thereof, and any combination thereof; Element 18: wherein the microsphere further comprises a cellulose ester; Element 19: Element 18 wherein the cellulose ester comprises at least one organic ester substituent selected from the group consisting of an C₁-C₂₀ aliphatic ester, a functional C₁-C₂₀ aliphatic ester, an aromatic ester, a substituted aromatic ester, any derivative thereof, and any combination thereof; Element 20: Element 18 wherein the cellulose ester comprises at least one inorganic ester substituent selected from the group consisting of hypochlorite, chlorite, chlorate, perchlorate, sulfite, sulfate, a sulfonate, fluorosulfate, nitrite, nitrate, phosphite, phosphate, phosphonates, borate, any derivative thereof, and any combination thereof; Element 21: wherein the microsphere further comprises at least one ester derivative of at least one selected from the group consisting of hemicellulose, algenates, chitosan, and any combination thereof; and Element 22: wherein the microsphere has a surface area of about 1 m²/g to about 50 m²/g as measured by BET with nitrogen gas. Exemplary combinations of embodiments may include, but are not limited to, Element 16 in combination with Element 17; at least one of Elements 16 and 17 in combination with Element 18 (optionally in combination with at least one of Elements 19 and 20); Element 18 in combination with at least one of Elements 19 and 20; Element 21 in combination with any of the foregoing; and Element 22 in combination with any of the foregoing.

Additional embodiments may include:

E: a cosmetic comprising the polysaccharide ester microsphere according to Embodiment D optionally including the corresponding Elements (or produced by a method of Embodiments A-C optionally including the corresponding Elements) that further comprises at least one of an aroma, a flavorant, and a colorant, wherein the cosmetic is at least one selected from the group consisting of a bronzer, a face powder, an eye shadow, an eye liner, a mascara, a blush, a brow powder, a baby powder, a lip gloss, and a lipstick;

F. an agricultural product comprising a polysaccharide ester microsphere according to Embodiment D optionally including the corresponding Elements (or produced by a method of Embodiments A-C optionally including the corresponding Elements) dispersed in a fluid, wherein the polysaccharide ester microsphere further comprises at least one selected from the group consisting of a herbicide, a fungicide, an insecticide, a bactericide, a nitrogen source, a growth promoter, and any combination thereof;

G. a cigarette filter that comprises a filter material and the polysaccharide ester microsphere according to Embodiment D optionally including the corresponding Elements (or produced by a method of Embodiments A-C optionally including the corresponding Elements) dispersed therein;

H. a cigarette filter that comprises a filter material and a cavity or capsule therein, wherein the polysaccharide ester microsphere of Embodiment G is contained in the cavity or capsule; and

I. a food product that comprises a polysaccharide ester microsphere according to Embodiment D optionally including the corresponding Elements (or produced by a method of Embodiments A-C optionally including the corresponding Elements) that further comprises at least one of a flavorant, an aroma, a food agent, and a nutritional supplement.

To facilitate a better understanding of the present invention, the following examples of preferred or representative embodiments are given. In no way should the following examples be read to limit, or to define, the scope of the invention.

### EXAMPLES

### Example 1

Cellulose diacetate microspheres loaded with Acid Blue 290 were prepared by including Acid Blue 290 in the dope used to produce the microspheres where the weight ratio of cellulose diacetate to Acid Blue 290 was about 9:2. The loaded microspheres were soaked in water, which was analyzed via spectroscopic techniques to determine the concentration of Acid Blue 290 released from the loaded microspheres and consequently a release rate. The average release rate was about 61 ppm dye/g of microspheres/hr. However, in experiments where the grams of microspheres were increase with the same volume of water, the concentration of the dye released into the water did not increase as expected, i.e., twice as many microspheres did not yield twice the concentration of dye in the water. Without being limited by theory, it is believed that additives that are soluble in or have an affinity for the microsphere compositions (e.g., cellulose diacetate) and the surrounding environment (e.g., water) can reach an equilibrium under static conditions.

### Example 2

Never dried cellulose diacetate microspheres prepared from a 90% acetic acid/10% water solvent and a room temperature precipitation bath were solvent exchanged with methanol. The microspheres were filtered and placed wet in an ibuprofen/methanol solution for 20.5 hours under agitation at room temperature. The microspheres were filtered and air dried at room temperature. This method produced microspheres with a 62.6% by weight loading of ibuprofen. The ibuprofen loaded microspheres were packed in a column. Water was flowed through the column at about 10-12 mL/min, and the effluent was analyzed for ibuprofen concentration. The loaded microspheres released the ibuprofen initially quickly, like a “burst release,” for approximately the first 30-45 minutes and then begins to level off, perhaps with a slow decline over time, to a rate of about 0.6-0.8 mg/min.

### Example 3

Cellulose diacetate microspheres were tested for absorption of limonin from navel orange juice, the component of navel orange juice that make is bitter. Hydrated microspheres (microsphere having be soaked in water to replace air in the void spaces) were added to samples of navel orange juice (about 2 g dry weight microspheres to 100 mL of navel orange juice), agitated for 1 hour, and then removed with a wire mesh. The microspheres absorbed about 80% of the limonin in the navel orange juice.

### Example 4

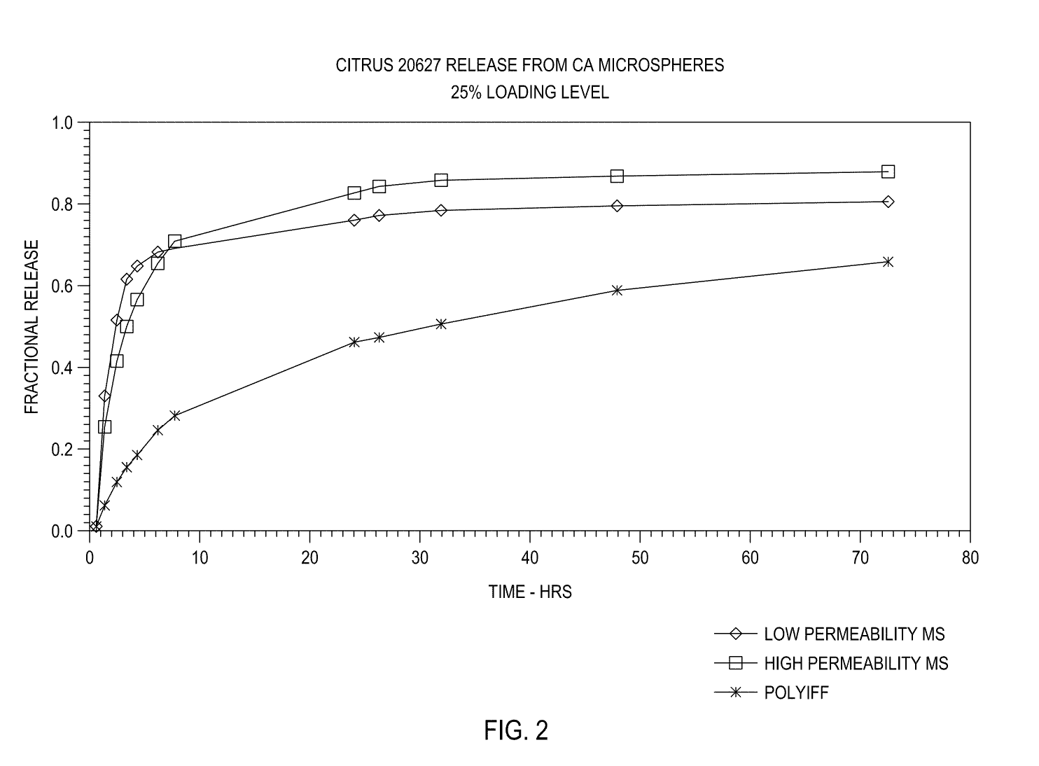

Cellulose diacetate microspheres were analyzed for fragrance release and compared to a polyethylene loaded with the same fragrance. The fragrance used was CITRUS 20627 (available from International Flavors & Fragrances). The microspheres were loaded to about 25% by weight with the fragrance, approximately that of the polyethylene loaded sample. Fragrance release was measured by weight loss. The results indicated that the fragrance released faster from the microspheres than the polyethylene, which may be due to the porous nature and higher surface area of the microspheres. As shown in FIG. 2, the microspheres released about 70% of the CITRUS 20627 in the first 8-10 hours, while the polyethylene, over the same time period, released about 30%. Over the next 70 hours, the microspheres continued a slow release of fragrance to about 80% released, and the polyethylene continued on a moderate release rate profile reaching about 60% release in the same time frame.

### Example 5

Flake of cellulose diacetate with molecular weight of about 75,000 g/mol was used to form a dope in 90 wt % acetic acid and 10 wt % water with a solids content of about 9 wt %. The dope was sprayed an anti-bearding fluid nozzle and ¼ J air cap (available from Spraying Systems Co.) into a 43.5 L coagulation bath of deionized water containing 110 mL TWEEN 80 (a polyethylene sorbitol ester surfactant, available from Sigma-Aldrich) and 10 mL SIGMA ANTIFOAM B (an aqueous silicone emulsion, available from Sigma-Aldrich) was at room temperature and about 115 cm to about 155 cm from the nozzle to produce microspheres.

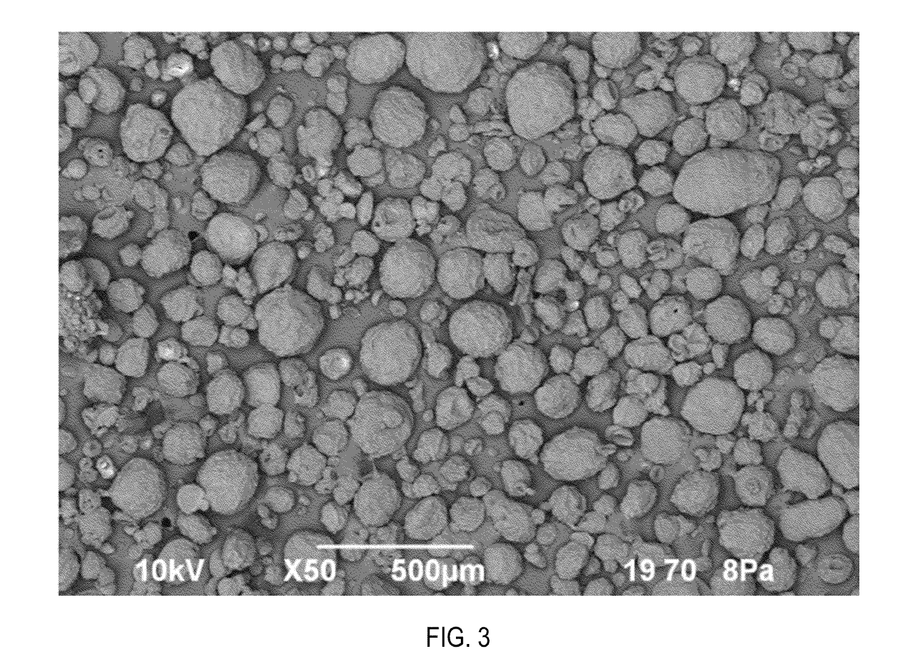

The resultant microspheres were analyzed for particle size via a sieve method (CA-MS2 only) (Table 1); particle size via a light scattering method using a Malvern Instrument Model 2000 (Table 2); surface area, total pore volume, and average pore size via a BET method using a Micrometrics ASAP 2020 Accelerated Surface Area and Porosimetry System (Table 3); and morphology via scanning electron microscopy (“SEM”) (FIGS. 3-5).

TABLE 1
(CA-MS2)

| Sieve Size | Approx. Microns | % by Wt |
|---|---|---|
| #60 | >250 | 4.7 |
| #80 | 180-250 | 15.9 |
| #100 | 150-180 | 21.9 |
| #140 | 106-150 | 29.3 |
| #230 | 63-106 | 23.4 |
| catch pan | <63 | 4.8 |

TABLE 2

| Sample | D₁₀ (microns) | D₅₀ (microns) | D₉₀ (microns) |
|---|---|---|---|
| CA-MS1 | 59.4 | 131.9 | 257.9 |
| CA-MS2 | 65.7 | 142.2 | 281.8 |
| CA-MS3 | 72.7 | 161.9 | 319.3 |
| CA-MS4 | 27.8 | 109.5 | 396.9 |

TABLE 3

| | BET Surface | Total Pore | Average Pore Size |
|---|---|---|---|
| Sample | Area (m²/g) | Volume (mL/g) | (angstroms) |
| CA-MS1 | 12.3 | 0.074 | 239 |
| CA-MS2 | 12.6 | 0.077 | 246 |
| CA-MS3 | 16.8 | 0.109 | 259 |

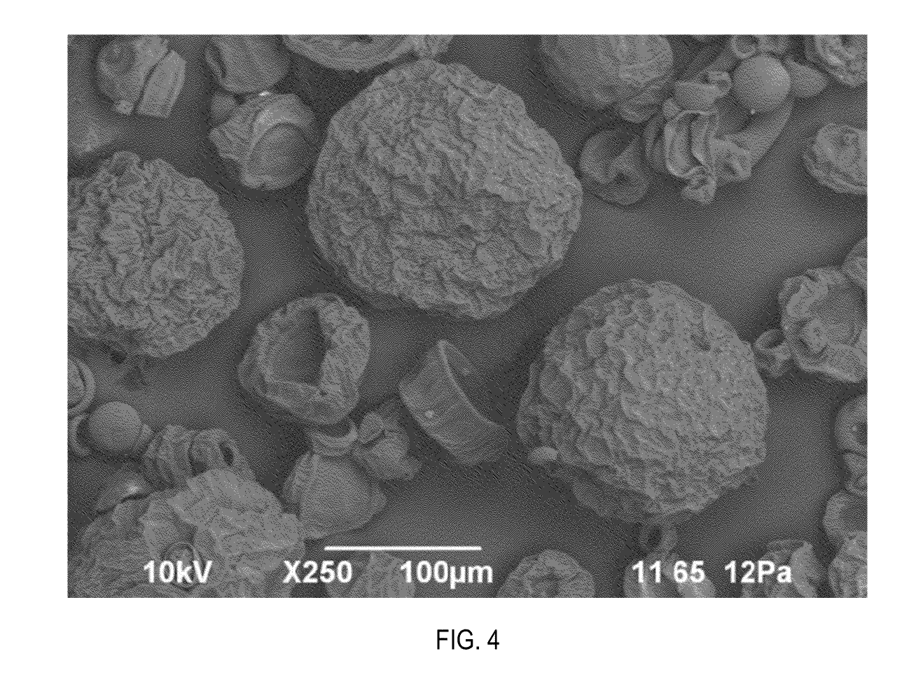

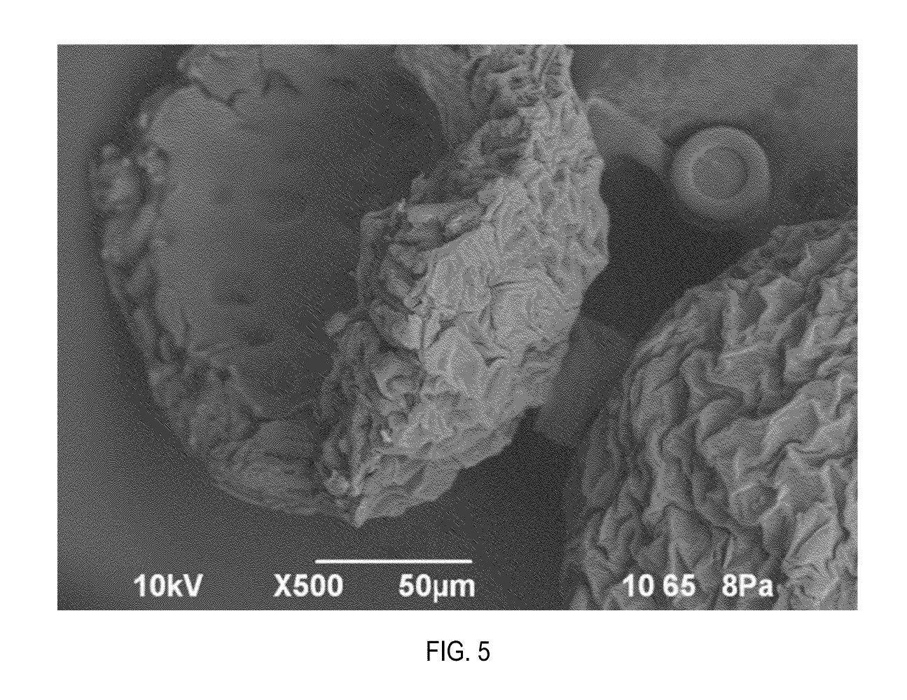

FIG. 3 provides an SEM micrograph of several microspheres from the CA-MS3 sample. The microspheres are substantially spherical. FIG. 4 provides a higher resolution SEM micrograph of the surface of a microsphere that shows a rippled or crinkled surface with no significant visible porosity. FIG. 5 provides a higher resolution SEM micrograph of the cross-section of a microsphere that shows hollow interior and evidence of porosity in the walls of the microsphere. The walls of the microsphere are measured at about 40 microns to about 55 microns.

### Example 6

Cellulose diacetate from a cellulose acetate product (i.e., directly from polysaccharide ester synthesis 1.1 of FIG. 1 without finishing) with molecular weight of about 75,000 g/mol was diluted with water and acetic acid to yield a dope of 90 wt % acetic acid and 10 wt % water with a solids content of about 9 wt %. No filtering was performed in the preparation of the dope. It should be noted that because filtration was not performed, the dope contains magnesium salts like magnesium sulfate and magnesium acetate.

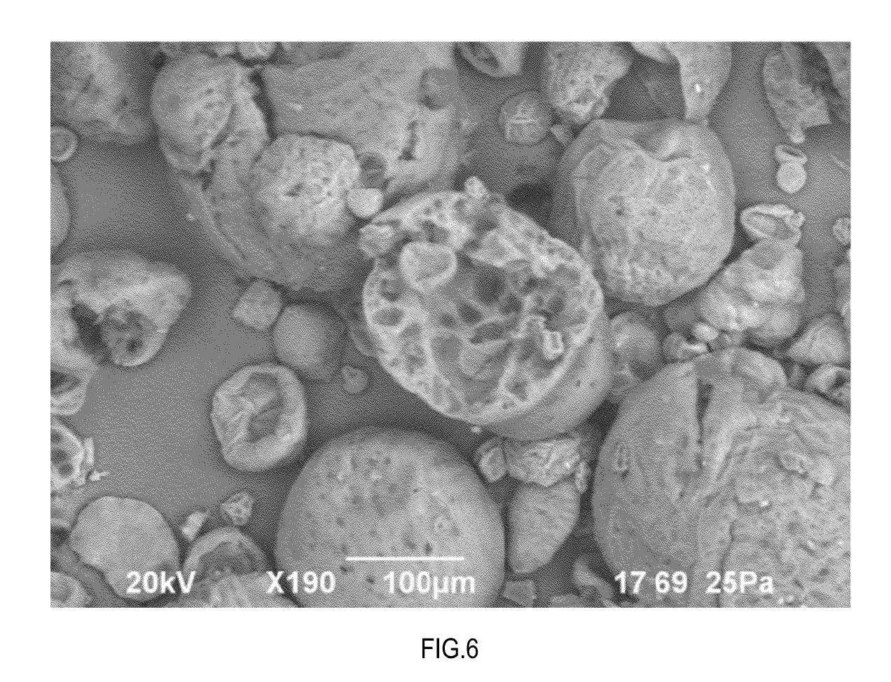

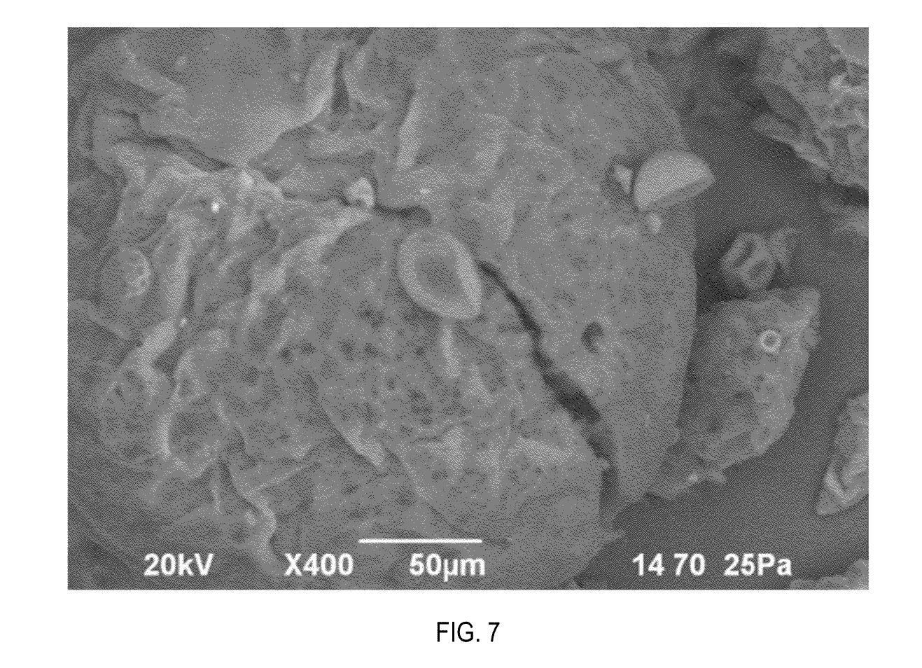

Microspheres were produced under the same conditions as Example 5. The resultant microspheres were analyzed for particle size via a sieve method (Table 4); particle size via a light scattering method (Table 5); surface area via a BET method (Table 6); and morphology via SEM (FIGS. 6-7), each as described above in Example 5.

TABLE 4

| | | % by Wt |
|---|---|---|
| Sieve Size | Approx. Microns | (CA-MS5) |
| #60 | >250 | 17.5 |
| #80 | 180-250 | 29.6 |
| #100 | 150-180 | 15.7 |
| #140 | 106-150 | 20.8 |
| #230 | 63-106 | 11.6 |
| catch pan | <63 | 4.0 |

TABLE 5

| Sample | D₁₀ (microns) | D₅₀ (microns) | D₉₀ (microns) |
|---|---|---|---|
| CA-MS5 | 46.0 | 126.3 | 249.0 |

TABLE 6

| | BET Surface |
|---|---|
| Sample | Area (m²/g) |
| CA-MS5 | 23.7 |

FIG. 6 provides an SEM micrograph of several microspheres produced in this example including a cross-section of a microsphere. The microspheres are substantially spherical, though not as uniform as the microspheres of Example 5. Similar to the microspheres of Example 5, these microspheres have a hollow center and walls similar in thickness. However, these microspheres have less dense, more porous walls as compared to the microspheres of Example 5. FIG. 7 provides a higher resolution SEM micrograph of the surface of a microsphere that shows smoother surface than the microspheres of Example 5 but with more visible porosity.

This example demonstrates that polysaccharide ester microspheres may be formed directly from polysaccharide ester product, which reduces capital costs, operating costs, and manufacturing time. Further, this example illustrates that the morphology, porosity, and, consequently, release rates of the polysaccharide ester microspheres may be altered with the inclusion of salts in the polysaccharide ester dope.

### Example 7

Starch diacetate in dried form (i.e., finished polysaccharide ester from FIG. 1) with molecular weight of about 16,000 g/mol was used to form a dope in 90 wt % acetic acid and 10 wt % water with a solids content of about 35 wt %.

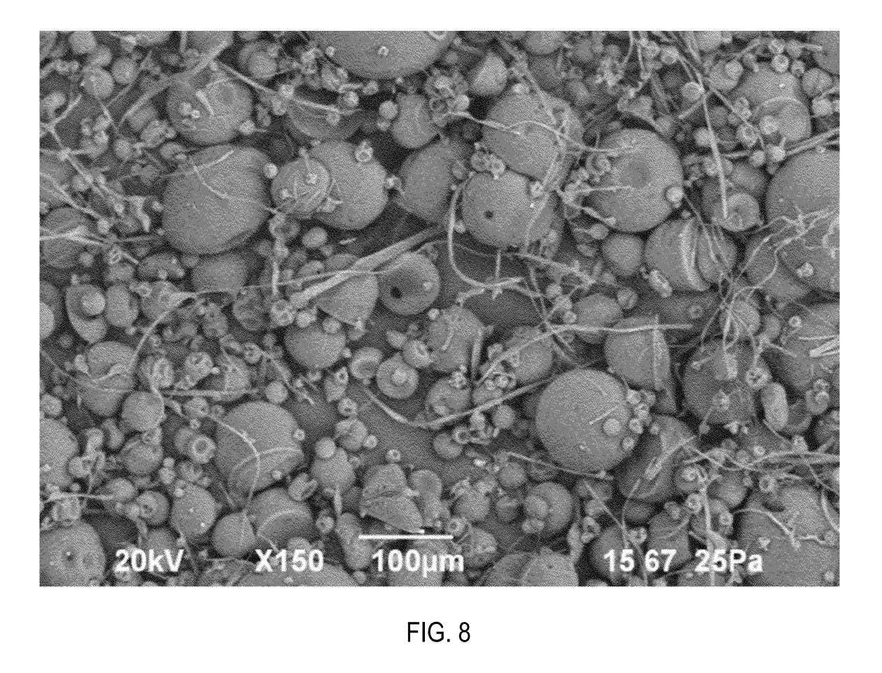

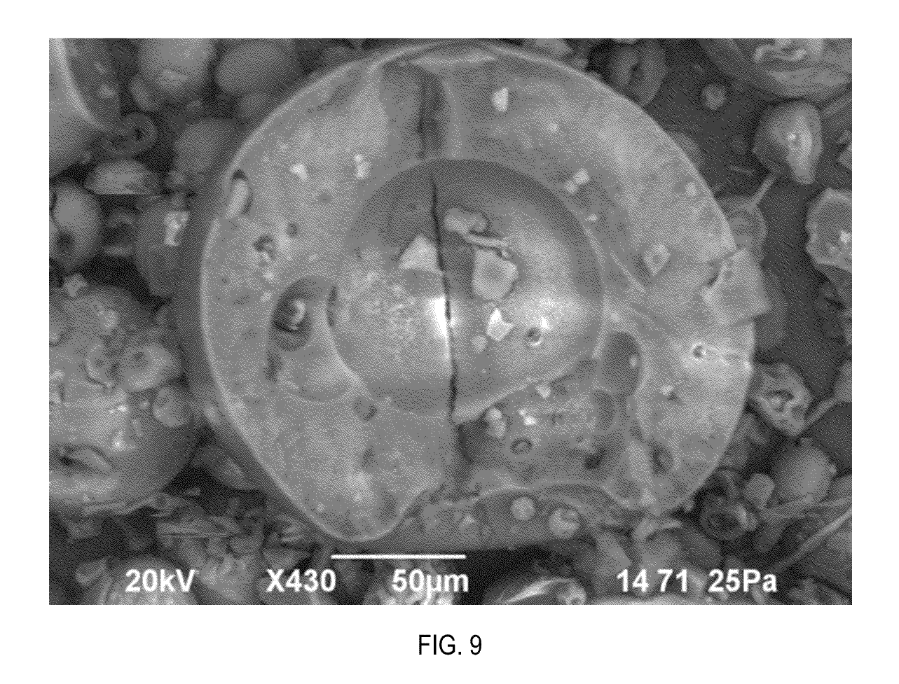

Microspheres were produced under the same conditions as Example 5 with a distance between the nozzle and the coagulation bath being about 63 cm to about 115 cm. The resultant microspheres were analyzed for particle size via a light scattering method (Table 7); surface area via a BET method (Table 8); and morphology via SEM (FIGS. 8-9), each as described above in Example 5.

TABLE 7

| Sample | D₁₀ (microns) | D₅₀ (microns) | D₉₀ (microns) |
|---|---|---|---|
| SA-MS1 | 79.4 | 165.4 | 299.0 |

TABLE 8

| | BET Surface |
|---|---|
| Sample | Area (m²/g) |
| SA-MS1 | 1.13 |

FIG. 8 provides an SEM micrograph of several microspheres produced in this example. The microsphere product appears to contain starch acetate microspheres of two different sizes (i.e., a bimodal diameter distribution) that are substantially spherical and fibers. The larger of the spheres are smaller than the cellulose acetate microspheres of Examples 5 and 6. FIG. 9 provides a higher resolution SEM micrograph of the cross-section of a microsphere that shows hollow interior and evidence of porosity in the walls of the microsphere, though the walls of the microsphere appear to be more dense than that of the cellulose acetate microspheres of Examples 5 and 6. Further, in FIG. 9, the surface of adjacent microspheres indicates that the surface of these starch acetate microspheres is significantly smoother than that of the cellulose acetate microspheres of Examples 5 and 6.

### Example 8

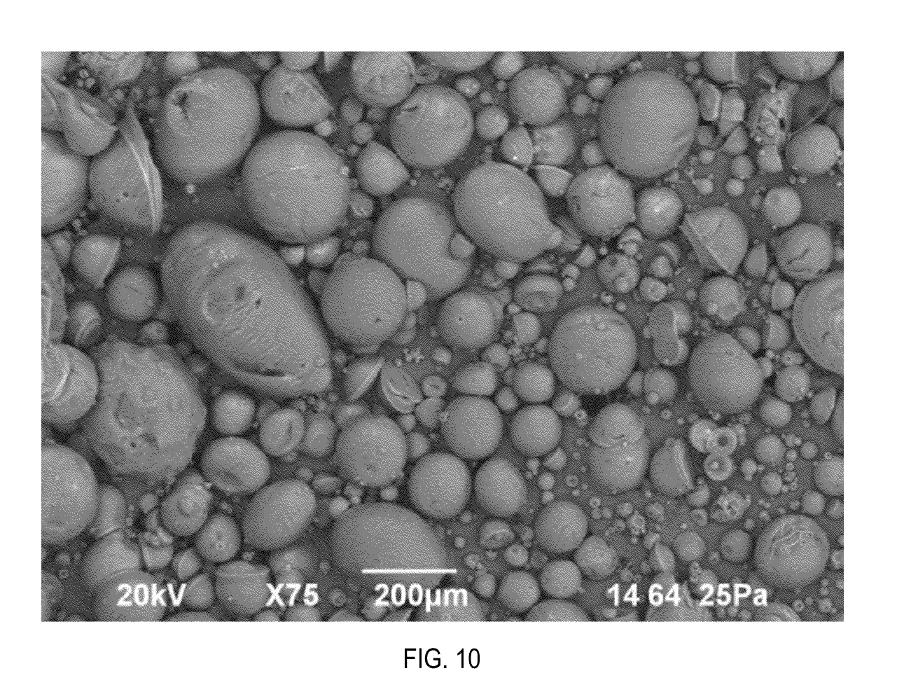

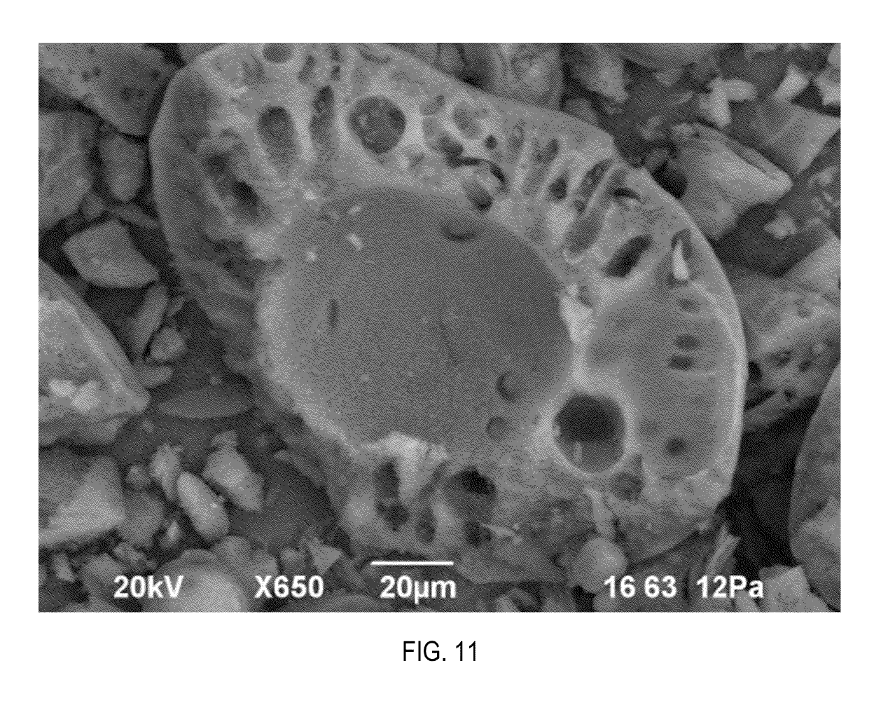

A dope comprising mixture by weight of starch triacetate and cellulose diacetate was produced by mixing equal volumes of a starch triacetate dope (about 35 wt % solids in 90 wt % acetic acid and 10 wt % water) and a cellulose diacetate dope (about 9 wt % solids in 90 wt % acetic acid and 10 wt % water), thereby producing a dope comprising about 80% starch triacetate and about 20% cellulose diacetate each by weight of the total polysaccharide ester. Microspheres were produced under the same conditions as Example 5 with a distance between the nozzle and the coagulation bath being about 63 cm to about 115 cm. The resultant microspheres were analyzed for morphology via SEM (FIGS. 10-11).

FIG. 10 provides an SEM micrograph of several microspheres in this example. The microsphere product appears to contain starch acetate microspheres with a broad diameter distribution that are substantially spherical to ovular. FIG. 10 provides a higher resolution SEM micrograph of the cross-section of a microsphere that shows hollow interior and evidence of porosity in the walls of the microsphere with a wall density between that of the cellulose acetate microspheres of Examples 5 and 6 and the starch acetate microspheres of Example 7. Further, in FIG. 10, the surface the starch acetate/cellulose acetate microspheres is significantly smoother than that of the cellulose acetate microspheres of Examples 5 and 6.

### Example 9

Flake of cellulose diacetate with a molecular weight of about 75,000 g/mol was used to form a dope in 90 wt % acetic acid and 10 wt % water with a solids content of about 9 wt %. Limonene flavor oil was dispersed in the dope using a rotor-stator homogenizer at greater than 15,000 rpm. Limonene at 50%, 60%, and 70% loading levels was dispersed in the dope. The dope was sprayed via an anti-bearding fluid nozzle and ¼ J air cap into a coagulation bath of deionized water containing TWEEN 80 and SIGMA ANTIFOAM B at room temperature and located about 115 cm to about 155 cm from the nozzle to produce microspheres.

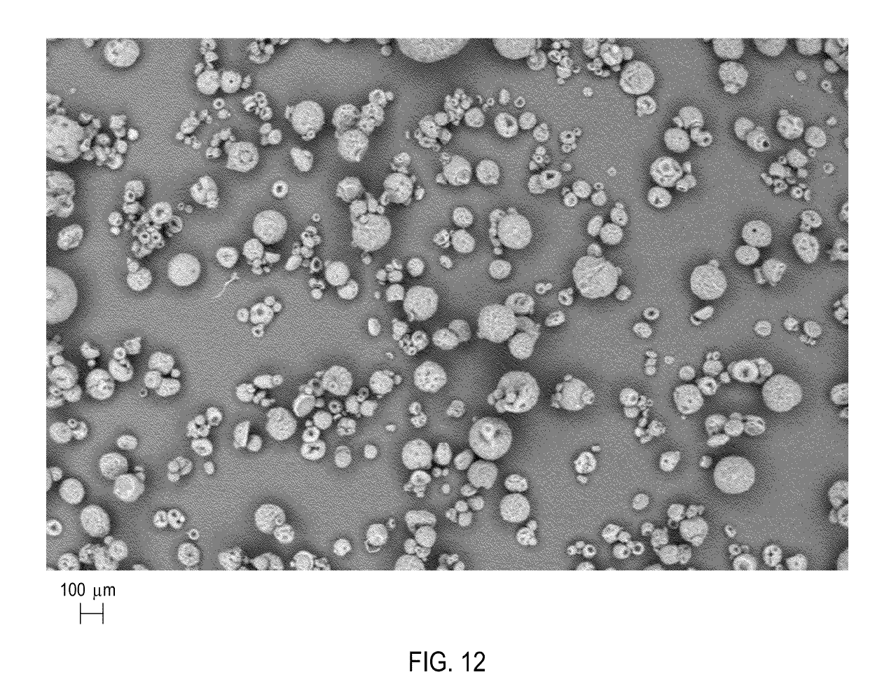

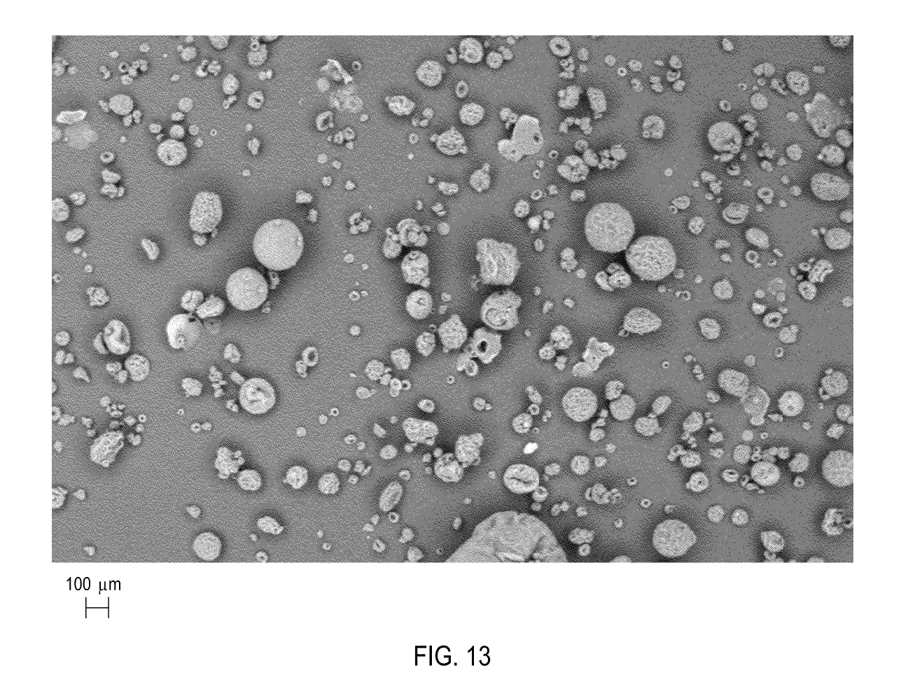

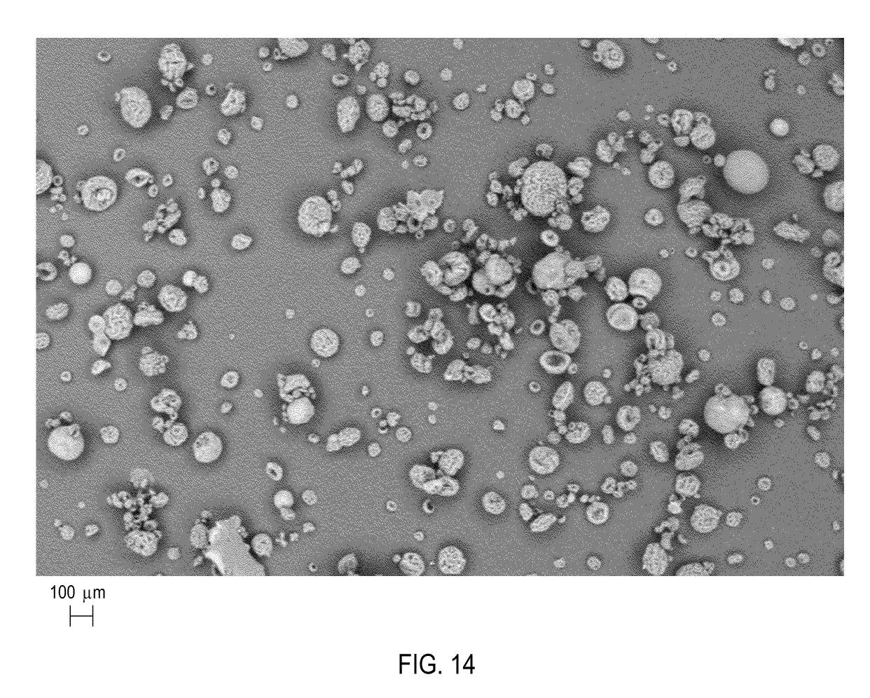

FIGS. 12-14 provide SEM micrographs of the 50%, 60%, and 70% limonene flavor oil loading levels, respectively. In each sample, the microspheres are substantially spherical and appear to be similar in structure to those of Example 5 (cellulose diacetate microspheres without limonene). However, the size of the microspheres appears to be less than the Example 5 microspheres, or at least the volume fraction of smaller microspheres appears to be higher.

### Example 10

Flake of cellulose diacetate with molecular weight of about 75,000 g/mol was used to form a dope in 90 wt % acetic acid and 10 wt % water with a solids content of about 9 wt %. Prior to cellulose diacetate flake addition, acesulfame potassium (SUNNETT®, a high intensity sweetener, available from Celanese) was fully dissolved in the acetic acid/water solvent at a solids content (cellulose diacetate weight basis) of about 200 wt %. The sweetener-loaded cellulose diacetate dope was sprayed via an anti-bearding fluid nozzle and ¼J air cap into a 43.5 L coagulation bath of deionized water containing 110 mL TWEEN 80 and 10 mL SIGMA ANTIFOAM B at room temperature and about 89 cm from the nozzle to produce microspheres.

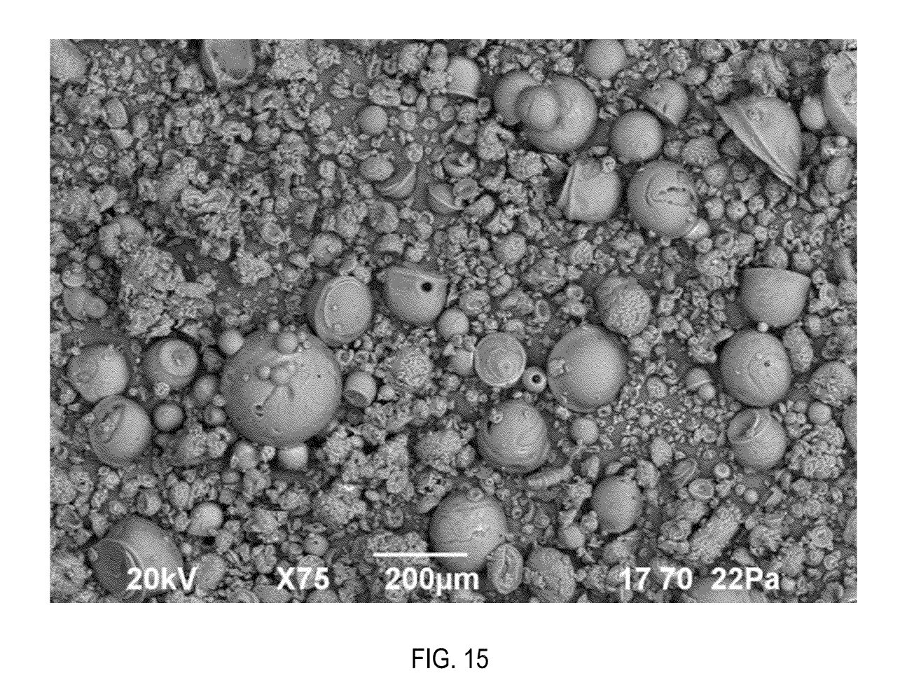

FIG. 15 provides an SEM micrograph of the resultant microspheres, which includes larger microspheres with a smoother surface and smaller microspheres with a surface that appears to be similar to those of Example 5.

### Example 11

Flake of cellulose diacetate with molecular weight of about 75,000 g/mol was used to form a dope in 90 wt % acetic acid and 10 wt % water with a solids content of about 9 wt %. Prior to cellulose diacetate flake addition, acesulfame potassium (SUNNETT®) was fully dissolved in the acetic acid/water solvent at a solids content (cellulose diacetate weight basis) of about 200 wt %. The sweetener-loaded cellulose diacetate dope was sprayed via an anti-bearding fluid nozzle and ¼ J air cap into a 19 L coagulation bath of denatured ethanol at room temperature and about 89 cm from the nozzle to produce microspheres.

The resultant microspheres were analyzed for particle size via a sieve method (Table 9). About 75 wt % of the microspheres are about 63 microns to about 150 microns, which is smaller than the cellulose diacetate microspheres of Example 5 that contain no acesulfame potassium where over 40 wt % are greater than about 150 microns.

TABLE 9

| Sieve Size | Approx. Microns | % by Wt |
|---|---|---|
| #60 | >250 | 4.7 |
| #80 | 180-250 | 6.0 |
| #100 | 150-180 | 10.3 |
| #140 | 106-150 | 45.0 |
| #230 | 63-106 | 24.7 |
| catch pan | <63 | 9.2 |

### Example 12

Flake of cellulose diacetate with molecular weight of about 75,000 g/mol was used to form a dope in 90 wt % acetic acid and 10 wt % water with a solids content of about 9 wt %. Prior to cellulose diacetate flake addition, a strawberry oil flavor (Mother Murphy's Laboratories Flavor # TK0746) was fully dissolved in the acetic acid/water solvent at a solids content (cellulose diacetate weight basis) of about 100 wt ° h. The flavor-loaded cellulose diacetate dope was sprayed via an anti-bearding fluid nozzle and ¼ J air cap into a 43.5 L coagulation bath of deionized water containing 110 mL TWEEN 80 and 10 mL SIGMA ANTIFOAM B at room temperature and about 89 cm from the nozzle to produce microspheres.

The cellulose diacetate microspheres produced from the dope containing strawberry oil were, by visual inspection, similar to that of the cellulose diacetate microspheres of Example 5 (no additional flavorants) and the cellulose diacetate microspheres of Example 10 (with acesulfame potassium sweetener).

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A microsphere comprising:
a starch ester; and
having a hollow core and a wall.

2. The microsphere of claim 1 further comprising a cellulose ester.

3. The microsphere of claim 1, wherein the microsphere has a surface area of about 1 m²/g to about 50 m²/g as measured by BET with nitrogen gas.

4. A cosmetic comprising the polysaccharide ester microsphere of claim 1 that further comprises at least one of an aroma, a flavorant, and a colorant, wherein the cosmetic is at least one selected from the group consisting of a bronzer, a face powder, an eye shadow, an eye liner, a mascara, a blush, a brow powder, a baby powder, a lip gloss, and a lipstick.

5. An agricultural product comprising a polysaccharide ester microsphere of claim 1 dispersed in a fluid, wherein the polysaccharide ester microsphere further comprises at least one selected from the group consisting of a herbicide, a fungicide, an insecticide, a bactericide, a nitrogen source, a growth promoter, and any combination thereof.

6. A cigarette filter that comprises a filter material and the polysaccharide ester microsphere of claim 1 dispersed therein.

7. A cigarette filter that comprises a filter material and a cavity or capsule therein, wherein the polysaccharide ester microsphere of claim 1 are contained in the cavity or capsule.

8. A food product that comprises a polysaccharide ester microsphere of claim 1 that further comprises at least one of a flavorant, an aroma, a food agent, and a nutritional supplement.
